(19) United States
(12) Patent Application Publication
(10) Pub. No.: US 2019/0298846 A1
(43) Pub. Date: Oct. 3, 2019

(54) CELL TARGETING CONJUGATES

(71) Applicant: LinXis B.V., Breda (NL)

(72) Inventors: Augustinus Antonius Maria Silvester Van Dongen, Utrecht (NL); Niels Jurriaan Sijbrandi, Utrecht (NL); Dennis Christian Johannes Waalboer, IJmuiden (NL); Hendrik Jan Houthoff, Amsterdam (NL)

(73) Assignee: LinXis B.V., Breda (NL)

(21) Appl. No.: 16/307,865

(22) PCT Filed: Jun. 6, 2017
(86) PCT No.: PCT/NL2017/050364

§ 371 Date: Dec. 6, 2018

(30) Foreign Application Priority Data
Jun. 6, 2016 (NL) 2016898

(51) Int. Cl. A61K 47/68; A61P 35/00; A61P 31/00

(52) U.S. Cl. CPC A61K 47/6803; A61P 31/00; A61P 35/00; A61K 47/6849

(57) ABSTRACT

Cell targeting conjugates that comprise a targeting moiety and one or more functional moieties bound thereto via a linker, wherein the linker comprises a transition metal complex and wherein at least 90% of the conjugates have a ratio of functional moieties to targeting moieties (DAR) of or less. A pharmaceutical composition comprising the cell targeting conjugates and the use of the conjugates and composition as a medicament, in particular, in the treatment of cancer.

### CROSS-REFERENCE TO RELATED APPLICATIONS

This application is a national phase entry under 35 U.S.C. § 371 of International Patent Application PCT/NL2017/050364, filed Jun. 6, 2017, designating the United States of America and published in English as International Patent Publication WO 2017/213494 A1 on Dec. 14, 2017, which claims the benefit under Article 8 of the Patent Cooperation Treaty to The Netherlands Patent Application Serial No. 2016898, filed Jun. 6, 2016.

### TECHNICAL FIELD

This disclosure relates to cell targeting conjugates comprising a targeting moiety and one or more functional moieties bound thereto via a linker, wherein the linker comprises a transition metal complex. The disclosure further relates to a pharmaceutical composition comprising the cell targeting conjugates and to the medical use of the pharmaceutical composition and the cell targeting conjugates.

### BACKGROUND

The vast majority of bioconjugation technologies available rely on the covalent coupling of organic linkers to proteins. Prime examples are linkers that incorporate a reactive ester or a maleimide for the coupling to lysine or cysteine residues, respectively. In the area of cell targeting conjugates (also referred to as antibody-drug conjugates or ADCs) both linkers have been successfully applied in the clinically approved ADCs KADCYLA® (ado-trastuzumab emtansine, T-DM1) and ADCETRIS® (brentuximab vedotin).

Despite some success achieved with ADCETRIS® and KADCYLA®, it is recognized by the experts in the field of cell targeting conjugates design and development that both types of linker applied in these cell targeting conjugates are of sub-optimal quality with regard to efficacy and tolerability of the cell targeting conjugates. Improved efficacy and improved tolerability of cell targeting conjugates in general is, therefore, aimed for in numerous development programs for linkers and conjugation strategies.

Next to the antibody and the payload (hereinafter referred to as functional moiety), such as a therapeutic compound or diagnostic compound, the linker system used is one of the three primary components of cell targeting conjugates that determine which cells are targeted, how the functional moiety (e.g., a cytotoxic drug) is released, and by which mechanism of action the cells will be killed in case the functional moiety is a drug. The linker system used can affect cell targeting conjugates stability and functional moiety release at several levels and, therefore, is of key importance for the efficacy and toxicity of cell targeting conjugates.

Firstly, the functional moiety (e.g., a cytotoxic drug) might be released from the antibody in the circulation after in vivo administration, resulting in sequestration of the functional moiety (e.g., a drug) in normal tissues.

Secondly, the antibody itself might be destabilized by the conjugation with one or more functional moiety (e.g., cytotoxic drugs), resulting in faster blood clearance of the complete conjugate, and sequestration in catabolic organs like liver and spleen. This phenomenon is, for example, observed upon stochastic conjugation of drugs to lysine and cysteine residues, which results in heterogeneous drug-to-antibody ratios (DAR) according to Poisson distribution, with more rapid clearance of conjugates of higher DAR (7-9).

Thirdly, after cellular uptake of the ADC by target-dependent or target-independent mechanisms, and subsequent catabolism, the functional moiety can be detached and eventually be released from the cell. If the functional moiety is a cytotoxic drug, it can subsequently kill neighboring cells, but also can cause toxicity, which can result in less predictable efficacy and tolerability.

Nowadays, to develop cell targeting conjugates with an improved therapeutic window developers of cell targeting conjugates focus on site-specific conjugation through the modification of antibodies by chemical means or protein engineering. However, the fast majority of cell targeting conjugates in clinical phase still rely on the conventional conjugation technologies, such as those described above. Several of the known methods for site-specific conjugation are 1) applying engineered cysteines or unnatural amino acids via mAb engineering, 2) applying enzymatic conjugation or 3) re-bridging reduced cysteines in the hinge region of the antibody.

All these three methods require modification of the antibody before the payload is conjugated to the antibody. Antibody modification may influence immunological properties of the antibody, resulting in undesired toxicities. Moreover, for a modified antibody regulatory hurdles with regard to market authorization are foreseeable. Hence, in view of this, it would be beneficial to obtain cell targeting conjugates that can be used in combination with antibodies for which regulatory approval has been (or will be) obtained. After all, unexpected toxicity problems with respect to the antibodies itself may be avoided. Moreover, the costs and time required for obtaining market authorization for using cell targeting conjugates comprising such an approved antibody will be considerably less.

Therefore, there is an urgent need for antibody conjugation technologies that provide for cell targeting conjugates with improved efficacy and improved pharmacokinetic properties. That is to say, uptake by the liver and reticuloendothelial system should be minimized. Immunoreactivity of the cell targeting conjugates provided by improved antibody conjugation technology should be low and both the drug-linker linkage and the antibody-linker linkage should be sufficiently stable. Furthermore, binding efficacy to its target and internalization of the conjugated antibody should be minimally affected upon conjugation, preferably not at all be affected.

### BRIEF SUMMARY

A first aspect of this disclosure, therefore, relates to cell targeting conjugates, which conjugates comprise a targeting moiety and one or more functional moieties bound thereto via a linker, wherein the linker comprises a transition metal complex and wherein at least 90% of the conjugates have a ratio of functional moieties to targeting moieties (DAR) of 4 or less.

With this disclosure, it has now been found that it is possible to prepare cell targeting conjugates that have a relatively narrow distribution profile of functional moieties to targeting moieties, i.e., in at least 90% of the conjugates the ratio between functional moieties and targeting moieties (e.g., antibodies) is 4 or less. This is an important aspect as it shows that only a limited number of amino acids of the targeting moiety is involved in the conjugation, which means that the conjugation is directed to specific amino acids.

With other types of linkers it is observed that the range of the ratios between functional moieties and targeting moieties is very broad, i.e., the DAR distribution of the antibody drug conjugate ranges from 0 till more than 8. Apparently these other types of linkers couple randomly to abundantly present amino acids of the targeting moiety (such as an antibody). Consequently the binding strength between such other types of linkers and the abundantly present amino acids may vary considerably. This gives rise to concerns as some of the functional moieties (i.e., cytotoxic drugs) may detach at a too early stage from the targeting moiety (e.g., an antibody). This leads to a reduced therapeutic window as only a relatively low dose of such a cell targeting conjugate can be used. Furthermore, if too many functional moieties bind to a targeting moiety (e.g., an antibody), the moiety may not reach the target site because the reticuloendothelial system removes it from the circulation. This problem has been solved with the cell targeting conjugates according to this disclosure.

A second aspect of this disclosure relates to cell targeting conjugates, which conjugates comprise a targeting moiety and one or more functional moieties bound thereto via a linker, wherein the linker comprises a transition metal complex and the targeting moiety an antibody and wherein at least 70% of the functional moieties are bound to the Fc part of the antibody.

It has surprisingly been found that the functional moieties are (via the linker) particularly linked to the Fc part of an antibody. This is very advantageous because it prevents that considerable amounts of functional moiety bind to the antigen binding sites of the antibodies, meaning that the sites for binding to the target (antigen binding sites) are not “clouded” with functional moieties preventing binding to the target site.

A third aspect of this disclosure relates to a pharmaceutical composition comprising a pharmaceutically acceptable carrier and the cell conjugates as described above.

A fourth aspect of this disclosure relates to cell targeting conjugates according to this disclosure or a pharmaceutical composition according to the disclosure for use as a medicament.

A fifth aspect of the disclosure relates to cell targeting conjugates according to the disclosure or to a pharmaceutical composition according to this disclosure for use in the treatment of cancer, preferably for use in the treatment of breast cancer or stomach cancer.

A sixth aspect of the disclosure relates to a method for the treatment of cancer, wherein the cancer preferably is breast cancer or stomach cancer, comprising administering to a patient in need thereof a pharmaceutically effective amount of the cell targeting conjugates according to the disclosure or a pharmaceutical composition according to the disclosure.

A last aspect of this disclosure relates to cell targeting conjugates, which conjugates comprise a targeting moiety and one or more functional moieties bound thereto via a linker, wherein the linker comprises a transition metal complex and wherein the cell targeting conjugates are used as a medicament.

### Definitions

The term “linker” as used herein generally has its conventional meaning and thus refers to a chemical moiety that forms a bridge-like structure between a targeting moiety and a functional moiety, such that the latter two are bound to each other.

The term “functional moiety” as used herein refers to a chemical group or molecule that has a certain biological, chemical, therapeutic and/or diagnostic function ex vivo or in vivo. Typical functional moieties are therapeutic compounds (i.e., drugs) or diagnostic compounds (i.e., tracers or dyes).

The term “targeting moiety” as used herein refers to a member of a specific binding pair, i.e., a member of a pair of molecules wherein one of the pair of molecules, has an area on its surface, or a cavity that specifically binds to, and is, therefore, defined as complementary with a particular spatial and polar organization of the other molecule, so that the pair have the property of binding specifically to each other. Examples of types of specific binding pairs are antigen-antibody, biotin-avidin, hormone-hormone receptor, receptor-ligand, enzyme-substrate, IgG-protein A.

The term “specific binding pair” as used herein refers to a member from a pair of molecules wherein one of the pair of molecules has an area on its surface or a cavity that specifically binds to, and is, therefore, defined as complementary with, a particular spatial and polar organization of the other molecule, so that the members of the pair have the property of binding specifically to each other. Examples of types of specific binding pairs are antigen-antibody, biotin-avidin, hormone-hormone receptor, receptor-ligand, enzyme-substrate, IgG-protein.

The term “targeted drug” as used herein refers to a drug coupled to a targeting moiety such as an antibody.

The term “immunoreactivity” as used herein has its normal scientific meaning and refers to the binding affinity of a member of a specific binding pair, such as a peptide, an antibody, an antibody fragment or nanobody.

The term “ratio of functional moieties to targeting moieties” as used herein relates to the number of functional moieties (such as cytotoxic drug molecules) that are bound (e.g., covalently or via a coordination bond) to a targeting moiety (e.g., an antibody). In the art the term “drug antibody ratio” or “DAR” is commonly used to designate this ratio between functional moieties and antibody. However, as is clear from the above, the targeting moiety according to the disclosure may, besides antibodies, also be, for example, a peptide, an antibody fragment or nanobody. However, for reasons of legibility also in relation to these types of targeting moieties (i.e., non-antibodies) the term “DAR” will be used in this disclosure.

### BRIEF DESCRIPTION OF THE DRAWINGS

FIG. 1. Schematic representation of the Lx conjugation technology and conjugation of DFO-Lx to trastuzumab.

FIG. 2. Analytical characterization of DFO-Lx-trastuzumab: Panel A: HPLC chromatogram at 280 nm, Panel B: LC-MS chromatogram, Panel C: Comparison of DAR distribution between DFO-Lx-trastuzumab (DAR 2.6) and DFO-trastuzumab (DAR 2.9; direct conjugation to lysines). Note that conjugation of DFO-Lx afford an ADC having a narrower DAR range implying less amino acids are involved in the conjugation.

FIG. 3. Chemical structures of a) AF-Lx-trastuzumab and b) AF-Mal-trastuzumab.

FIG. 4. Assessment of DFO-Lx binding throughout mAb molecule: a) SDS-PAGE followed by phosphor imager analysis of non-reduced and reduced DFO-Lx-trastuzumab (lanes 1 and 2, respectively) and non-reduced and reduced DFO-trastuzumab (lanes 3 and 4, respectively). Note: preferential binding of DFO-Lx to heavy chain, b) the distribution of the payload after DTT reduction or pepsin or papain digestion. Note: preferential binding of DFO-Lx to the Fc fragment.

FIG. 5. Biodistribution of ⁸⁹Zr-DFO-Lx-trastuzumab (gray bars) and ⁸⁹Zr-DFO-trastuzumab (black bars) at 72 hours after injection in NCI-N87 tumor-bearing mice.

FIG. 6. In vitro cell viability assay. The effect of AF-Lx-trastuzumab (black square), AF-Mal-trastuzumab (triangle up), ado-trastuzumab emtansine (triangle down) and AF-Lx-IgG-B12 (circle) on the viability of a) NCI-N87, b) MDA-MB-231, c) JIMT-1 and d) comparison of the cell viability of the drug-linkers AF-Lx (black square open) and AF-Mal (triangle open) with their corresponding ADCs AF-Lx-trastuzumab (black square closed) and AF-Mal-trastuzumab (triangle closed), respectively, on NCI-N87.

FIG. 7. The effect of AF-Lx conjugation on trastuzumab biodistribution characteristics in NCI-N87 tumor bearing mice: a) blood kinetics of AF-Lx-trastuzumab-⁸⁹Zr (black square), AF-Mal-trastuzumab-⁸⁹Zr (triangle) and trastuzumab-⁸⁹Zr (circle), b) biodistribution at 96 hours p.i. of AF-Lx-trastuzumab-⁸⁹Zr (black bars), AF-Mal-trastuzumab-⁸⁹Zr (light-gray bars) and ⁸⁹Zr-trastuzumab (dark-gray bars), and c) PET-CT image of a NCI-N87 tumor bearing mouse 96 hours after injection of AF-Lx-trastuzumab-⁸⁹Zr.

FIG. 8. Therapeutic efficacy of AF-Lx-trastuzumab in a) NCI-N87 tumor bearing mice b) JIMT-1 tumor bearing mice. Legend: vehicle (grey line, star), trastuzumab (diamond), ado-trastuzumab emtansine (triangle down), AF-Lx-trastuzumab, AF-Lx-trastuzumab 15 mg/kg (black line, closed square), AF-Lx-trastuzumab 30 mg/kg (black line, open square), AF-Lx-IgG-B12 (circles), AF-Mal-trastuzumab 15 mg/kg (closed triangle up) and AF-Mal-trastuzumab 15 mg/kg (open triangle up).

FIG. S1. Consistency of DFO-Lx coupling to different mAbs. LC-MS chromatogram of a) DFO-Lx-trastuzumab (DAR 3.2), b) DFO-Lx-obinutuzumab (DAR 2.9) and c) DFO-Lx-ofatumumab (DAR 3.0).

FIG. S2. Assessment of DFO-Lx binding throughout mAb molecule: DAR determination of heavy chain and light chain by SEC-MS for DFO-Lx-trastuzumab and DFO-trastuzumab.

FIG. S3. Assessment of AF-Lx binding throughout mAb molecule: SEC-MS of AF-Lx-trastuzumab a) intact, b) DTT reduced, c) pepsin reduced and d) papain reduced.

FIG. S4. In vitro serum stability of a)⁸⁹Zr-DFO-Lx-trastuzumab, and b)⁸⁹Zr-DFO-trastuzumab, as evaluated by radio-TLC, radio-HPLC, SDS-PAGE followed by phosphor imager analysis, and a binding assay according to Lindmo et al.

FIG. S5. Kaplan-Meier survival curves of a) NCI-N87 tumor bearing mice, b) JIMT-1 tumor bearing mice. Legend: vehicle (grey line, star), trastuzumab (diamond), T-DM1 (triangle down), AF-Lx-trastuzumab 15 mg/kg (black line, closed square), AF-Lx-trastuzumab 30 mg/kg (black line, open square), AF-Lx-IgG-B12 (circles), AF-Mal-trastuzumab 15 mg/kg (closed triangle up) and AF-Mal-trastuzumab 15 mg/kg (open triangle up).

### DETAILED DESCRIPTION

A first aspect of the disclosure relates to cell targeting conjugates, which conjugates comprise a targeting moiety and one or more functional moieties bound thereto via a linker, wherein the linker comprises a transition metal complex and wherein at least 90% of the conjugates have a ratio of functional moieties to targeting moieties (DAR) of 4 or less (i.e., of 1 up to and including 4).

Because of the narrow and constant distribution profile one can conclude that only a small number of amino acids of the targeting moiety is involved in the coupling with the linker. Furthermore, the link between these specific amino acids and the linker is strong. This means that after in vivo administration the functional moiety (which is connected to the targeting moiety via the linker) will not release from the targeting moiety whilst it is in circulation. Hence, the sequestration of the functional moiety (e.g., a cytotoxic drug) in normal tissue is avoided. Furthermore, site restricted conjugation is important because if the functional moieties can be linked to too many different amino acids, the immunoreactivity of the targeting moiety (e.g., an antibody) will be affected by the presence of the functional moieties. After all, in such a case too many functional moieties may bind to one targeting moiety (i.e., the DAR will become too high), which may considerably reduce the immunoreactivity of the targeting moiety.

In view of the advantageous properties of the conjugates of this disclosure, one is able to achieve a broad therapeutic window.

Furthermore, due to the fact that the linker comprises a transition metal complex (such as platina), the targeting moiety (such as an antibody) requires no modification for facilitating the coupling between the linker and the targeting moiety. Hence, the targeting moiety can remain in a form for which a separate marketing authorization has been provided or is applied for. Consequently, the regulatory approval time for the cell-targeting conjugate as a whole may be reduced significantly as one does not need to examine changes made to the targeting moiety (e.g., an antibody).

Preferably, at least 50% of the conjugates have a ratio of functional moieties to targeting moieties (also referred to as DAR) of 2 or 3. It has been found that if the conjugates comprise 2 to 3 functional moieties per targeting moiety (such as an antibody) the immunoreactivity of the targeting moiety remains excellent and that sufficient functional moieties (such as a cytotoxic drug) is supplied to the tissue of interest.

The functional moiety is in a preferred embodiment of the disclosure a therapeutic compound, a diagnostic compound or a chelating agent.

It is particularly preferred when the functional moiety is a therapeutic compound that inhibits a signal transduction cascade in a cellular system, interferes with the cytoskeleton or intercalates in the DNA double helix. It is further preferred that the functional moiety has anti-inflammatory, anti-hypertensive, anti-fibrotic, anti-angiogenic, anti-tumor, immune-stimulating or apoptosis-inducing activity, preferably the therapeutic compound has an anti-tumor activity.

According to the disclosure, the functional moiety may be a therapeutic compound chosen from the group of kinase inhibitors, or pro-drugs thereof. In another embodiment of the disclosure, the kinase inhibitor is erlotinib, gefinitib, imatinib, pentoxifylline, PDTC, PTKI, SB202190, vatanalib, LY364947, Y27632, AG1295, SP600125.

Alternatively, the functional moiety chosen is an angiotensin receptor blocker, such as losartan.

Alternatively, the functional moiety is a recombinant protein, such as TNF-related apoptosis-inducing ligand (TRAIL). Alternatively, the functional moiety is a therapeutic radionuclide, such as the beta emitters 90Y or 177Lu, or the alpha emitter 211At.

Alternatively, the functional moiety is a (super-)toxic drug chosen from the group of taxanes, anthracyclines, vinca alkaloids, calicheamicins, maytansinoids, auristatins, tubulysins, duocarmycins, amanitines or pyrrolo-benzodiazapine analogs.

Besides using therapeutic compounds as the functional moiety, also diagnostic compounds can be used. In an alternative embodiment the functional moiety is a fluorescent dye, such as IRDye800CW, DY-800, DY-831, Alexa fluor 750, Alexa fluor 790, and indocyanine green.

Other diagnostic compounds, which may be used in this disclosure as functional moiety, are radionuclides, PET-imagable agents, SPECT-imagable agents or MRI-imagable agents.

It is also possible to couple chelating agents as a functional moiety via the linker to the targeting moiety. These chelators may prior or after coupling to the targeting moiety be loaded with therapeutic or diagnostic radionuclides or non-radioactive metals. Possible chelating agents are EDTA, DPTA and desferioxamine (DFO). However, also macrocyclic chelating agents may be used, such as DOTA or p-SCN-Bn-DOTA, to which a transition metal PET radioisotope, a non-radioactive metal, or transition metal SPECT radioisotope, such as ^{99m}Tc or ¹⁹⁵mPt is coupled.

Alternatively, more than one kind of functional moiety is used. This way it is possible to bind different functional moieties, e.g., different useful combinations of therapeutic compounds or different combinations of useful diagnostic compounds to one targeting moiety. This way, a preferred combination of therapeutic compounds can be delivered to the tissue of interest.

In order to obtain a bond with adequate stability for in vivo applications it is preferred that the targeting moiety and/or the functional moiety comprise one or more sulphur-containing reactive sites and/or one or more nitrogen containing sites. It is particularly preferred that the functional moiety, such as a therapeutic compound, comprises one or more sulphur-groups and/or one or more nitrogen groups, preferably heterocyclic or aliphatic amines or aromatic nitrogen groups.

The targeting moiety is preferably a peptide, an antibody, an antibody fragment or a nanobody.

The targeting moiety preferably comprises a member of a specific binding pair and is thus able to bind to distinctive parts of certain cells or tissues. This way the targeting moiety is able to bring the functional moiety, which is attached thereto via the linker, to the place of interest.

The targeting moiety may comprise antibodies, such as monoclonal antibodies, derivates or fragments thereof or may comprise peptides.

A derivative of an antibody is defined herein as an antibody that has been altered such that at least one property—preferably an antigen-binding property—of the resulting compound is essentially the same in kind, not necessarily in amount. A derivative is provided in many ways, for instance through conservative amino acid substitution, whereby an amino acid residue is substituted by another residue with generally similar properties (size, hydrophobicity, etc.), such that the overall functioning is likely not to be seriously affected.

A fragment of an antibody is defined as a part that has at least one same property as the antibody in kind, not necessarily in amount. The functional part is capable of binding the same antigen as the antibody, albeit not necessarily to the same extent. A fragment of an antibody preferably comprises a single domain antibody (also referred to as nanobody), a single chain antibody, a single chain variable fragment (scFv), a Fab fragment or a F(ab′)2 fragment. Suitably, the targeting moiety is a monoclonal antibody, most preferably a monoclonal antibody chosen from the group of antibodies that have shown a capacity for selective tumor targeting, such as adalimumab, bevacizumab, catumaxomab, cetuximab, gemtuzumab, golimumab, infliximab, panitumumab, rituximab and trastuzumab or combinations thereof.

Alternatively, the targeting moiety is an antibody fragment, such as a therapeutic FAB, such as ranibizumab, a diabody, a minibody, a domain antibody, an affibody, a nanobody, such as ALX-0651, or an anticalcin.

The linker preferably comprises a platinum complex. The platinum complex may be a trans-platinum complex or it may be a cis-platinum complex. The cis-platinum complex is preferred and comprises preferably an inert bidentate moiety as a stabilizing bridge. In another embodiment the (platinum) complex comprises a tridentate moiety as a stabilizing bridge.

The relatively low functional moiety to targeting moiety ratio (DAR) of 4 or less of the cell targeting conjugates of the disclosure comprising such a linker contributes to an excellent immunoreactivity of the cell targeting conjugates, according to the disclosure. For example, bifunctional platinum(II)-complex ethylenediamine platinum(II) dichloride (hereinafter also referred to as “Lx” linker), a linker comprising a platinum complex and further comprising an inert bidentate moiety, was successfully applied in the improved cell targeting conjugates of the disclosure.

For example, a stable and efficaciously targeting cell targeting conjugate of the disclosure is the conjugate comprising mAb trastuzumab conjugated with 4-nitrobenzo-2-oxa-1,3-diazole (NBD) fluorophore through bifunctional platinum(II)-complex ethylenediamineplatinum(II) dichloride linker.

As said before, it is one of the many advantages of the antibody-drug conjugates of the disclosure that there is no need for modifying the targeting moiety, e.g., a peptide, an antibody or a fragment thereof, or nanobody before the functional moiety is coupled through the linker, according to the disclosure. Since modifying amino-acid side chains of a peptide or an antibody bears the risk for conformational changes in the peptide, nanobody or antibody, modification thereof may result in loss of targeting capacity of the peptide, antibody or nanobody. Furthermore, if the modification is in the region of the binding site of the targeting moiety, modifying a peptide, an antibody or nanobody can result in reduced affinity for the target cell, or even loss of targeting capacity.

Therefore, in one embodiment, the disclosure relates to cell targeting conjugates according to the disclosure, wherein the peptide, antibody, antibody fragment thereof or nanobody has not been modified for introducing a coupling site for a linker in the peptide, antibody, antibody fragment thereof or nanobody.

Surprisingly, it was found that the high and beneficial stability of the cell targeting conjugates of the disclosure, in addition to the beneficial highly maintained cell targeting capacity of the targeting moiety of the cell targeting conjugates of the disclosure, wherein the targeting moiety is an antibody, is due to the functional moiety predominantly binding to the heavy chain of the antibody through the conjugated linker, preferably the so-called “Lx linker.” That is to say, cell targeting conjugates of the disclosure wherein the targeting moiety is an antibody, are particularly effective in cell targeting and delivering functional moiety at the intended site of a target cell (e.g., a cancer cell), due to amongst others the location of the conjugation sides of the antibody and the functional moieties, which are predominantly at the heavy chain of the antibody. It was found that at least 89% of the functional moieties are bound to the heavy chain of the antibody in the cell targeting conjugates of the disclosure. In addition, it was found that at least 87% of the functional moieties are bound to the Fc portion of the heavy chain of the antibody in the cell targeting conjugates of the invention.

Thus, it is part of the disclosure that the cell targeting conjugates of the disclosure having an antibody as the targeting moiety, have a low DAR of 4 or less, wherein the functional moieties are predominantly bound to the heavy chain of the antibody, preferably predominantly bound to the Fc portion of the heavy chain. Hence, in an embodiment of this disclosure, at least 70%, preferably at least 80% of the functional moieties are bound to the Fc part of the antibody used as targeting moiety.

In view of the above, a second aspect of the disclosure relates to cell targeting conjugate, which conjugates comprise a targeting moiety and one or more functional moieties bound thereto via a linker, wherein the linker comprises a transition metal complex and the targeting moiety an antibody and wherein at least 70% of the functional moieties are bound to the Fc part of the antibody.

In a preferred embodiment of the disclosure, at least 80%, preferably at least 85% of the functional moieties are bound to the Fc part of the antibody.

In relation to these cell targeting conjugates, it is explicitly noted that the targeting moiety, the functional moiety and the linker—and other features of the cell targeting conjugates—maybe as has been described above in relation to the first aspect of the disclosure.

A third and fourth aspect of the disclosure relates to a pharmaceutical composition comprising the cell targeting conjugates (or mixture thereof) as has been described above in relation to the first and second aspect of the disclosure. The pharmaceutical composition comprises in addition a pharmaceutically acceptable carrier.

In accordance with the disclosure, the term “pharmaceutical composition” relates to compositions comprising the stable cell targeting conjugates as described hereinabove. Such pharmaceutical compositions comprise a therapeutically effective amount of these stable cell targeting conjugates and a pharmaceutical acceptable carrier.

These pharmaceutical compositions may be administered with a physiologically acceptable carrier to a patient. The term “carrier” as used herein refers to a diluent, adjuvant, excipient, or vehicle with which the therapeutic is administered. Such pharmaceutical carriers can be sterile liquids, such as water. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents.

These pharmaceutical compositions can take the form of solutions, suspensions, emulsion, tablets. The composition can be formulated as a suppository, with traditional binders and carriers such as triglycerides.

Examples of suitable pharmaceutical carriers are described in “Remington's Pharmaceutical Sciences” by E. W. Martin. Such compositions will contain a therapeutically effective amount of the cell targeting conjugates, together with a suitable amount of carrier so as to provide the form for proper administration to the patient. The formulation should suit the mode of administration.

In another preferred embodiment, the pharmaceutical composition is formulated in accordance with routine procedures as a pharmaceutical composition adapted for intravenous administration to human beings. Typically, compositions for intravenous administration are solutions in sterile isotonic aqueous buffer. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Pharmaceutical grade organic or inorganic carriers and/or diluents suitable for oral and topical use can be used to make up compositions containing the therapeutically active compounds. The compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

A fifth aspect of the disclosure relates to the above-mentioned cell targeting conjugates or pharmaceutical compositions for use as a medicament. The conjugates and pharmaceutical compositions thereof are particularly suitable for use in the treatment of cancer, in particular breast cancer and/or stomach cancer.

A sixth aspect of the disclosure relates to a method of treatment of cancer, which method comprises the administering of a patient in need thereof of a pharmaceutically effective amount of the cell targeting conjugates or pharmaceutical composition thereof. The method is particularly suitable for use in the treatment of breast cancer and/or stomach cancer.

A last aspect of the disclosure relates to cell targeting conjugates, which conjugates comprise a targeting moiety and one or more functional moieties bound thereto via a linker, wherein the linker comprises a transition metal complex and wherein the cell targeting conjugates are used as a medicament. The cell targeting conjugates are in particular suitable for use in the treatment of cancer, in particular breast cancer or stomach cancer. The linker preferably comprises a platinum complex. The targeting moiety is preferably an antibody. In this regard it is explicitly noted that the cell targeting conjugates according to this aspect of the disclosure, may be provided in the same preferred and alternative embodiments as have been described above in relation to the other aspects of the disclosure or as has been described in the examples below.

This disclosure will now be illustrated further by means of the following non-limiting examples.

### EXAMPLES

### Materials and Methods

### General Materials

Cell lines used were the breast cancer lines MDA-MB231, JIMT, BT-474 and SKBR3, the ovarian cancer cell line SKOV, and the gastric cancer cell line NCI-N87. JIMT-1 was obtained from DSMZ Germany on Mar. 19, 2012, after cytogenetic testing, and used within 6 months after resuscitation. NCI-N87 was obtained from ATCC United Kingdom on Feb. 29, 2012, after cytogenetic testing, and used within 6 months after resuscitation. SKBR3 was obtained from Dr. T. Oude Munnink (Department of Medical Oncology, University Medical Center Groningen, Groningen, The Netherlands), MDA-MB-231 from Roche, and SK-OV-3 from the Department of Medical Oncology, VU University Medical Center Amsterdam. All cell lines were checked for primary growth characteristics (morphology and growth rate) and HER2 expression. MDA-MB231 is a cell line with low HER2 expression; SKBR3, BT-474, SKOV-3 and NCI-N87 are overexpressing HER2; and JIMT-1 is developed from tumor cells of a patient with trastuzumab resistance and is HER2-positive (27) [“2004, Tanner”]. All starting reagents and solvents were obtained from Sigma-Aldrich or Fisher Scientific and used without further purification unless otherwise stated. Auristatin F (AF) was obtained from Concortis (San Diego, USA). Desferrioxamine (DFO), trastuzumab, obinituzumab, ofatumumab, cetuximab and KADCYLA® (T-DM1) were obtained from the hospital pharmacy. ⁸⁹Zr (≥0.15 GBq/nmol in 1 mol/L oxalic acid) was obtained from Cyclotron BV, Amsterdam. Water was distilled and deionized (18 MΩ/cm) by means of a MILLI-Q® water filtration system (Millipore, USA).

Conjugations of Lx-Based ADCs and Benchmarks and Labeling with ⁸⁹Zr

The Lx complexes DFO-Lx and AF-Lx were conjugated to different mAbs, essentially as described in the International patent application PCT/NL2016/050163 (which is herewith incorporated by reference).

Conjugation of DFO-Lx and AF-Lx to mAbs

Trastuzumab (71.0 μL, 21 mg mL⁻¹) was diluted with tricine buffer (12.3 μL, 200 mM, pH 8.5) and the platinum complex (40.0 μL, 5 mM stock solution) was added. The sample was incubated at 37° C. for 24 hours in a thermomixer at 550 rpm after which a thiourea solution (123.3 μL, 20 mM) in H₂O was added and the mixture was incubated for another 30 minutes at 37° C. The conjugate was purified using an AMICON® Ultra-15 centrifugal filter unit (30 kD MWCO, Merck Millipore) with PBS as solvent. Obinituzumab, ofatumumab and IgG-B12 were buffer-exchanged and concentrated to 21 mg·mL⁻¹ in a tricine buffer (20 mM, pH 8.5) before conjugation using AMICON® Ultra-15 centrifugal filter units.

Conjugation of AF-Mal to mAbs

AF-Mal was conjugated to different mAbs. Trastuzumab (71.0 μL, 21 mg mL⁻¹) was diluted with H₂O (100 μL) and borate buffer (38.8 μL, 250 mM sodium borate, 250 mM NaCl and 10 mM diethylenetrianinepentaacetic acid, pH 8.0) and tris(2-carboxyethyl)phosphine(TCEP) (3.3 equiv., 13.8 μL, 10 mM in H₂O) was added. The sample was incubated at 37° C. for 2 hours in a thermomixer at 550 rpm, after which the AF-Mal solution (6 equiv., 20.6 μL, 10 mM in DMSO) was added and the mixture was incubated for another 60 minutes at 0° C. Finally, the conjugation was quenched with N-acetyl-cysteine (5 μL, 100 mM) for 5 minutes at 0° C. and purified using an AMICON® Ultra-15 centrifugal filter unit (30 kD MWCO, Merck Millipore) with PBS as solvent.

Production of AF-Lx-Trastuzumab-⁸⁹Zr and AF-Mal-Trastuzumab-⁸⁹Zr

Conjugation of the activated ester of the succinylated DFO to the lysine residues in the mAb was carried out according a procedure described by Verel et al. (26) [“2003, Verel”]. The desired DFO to mAb ratio was obtained with a conjugation efficiency of 50%. AF-Lx-trastuzumab-⁸⁹Zr and AF-Mal-trastuzumab-⁸⁹Zr was obtained by first pre-modifying trastuzumab with DFO (DFO:trastuzumab ratio of 1), subsequent conjugation of AF-Lx or AF-Mal to the pre-modified trastuzumab according the methods described above, and finally labeling of the conjugates with ⁸⁹Zr according to a procedure described by Verel et al. (26) [“2003, Verel”].

### Analytical Procedures Used

### UV

Protein concentration was determined by UV spectroscopy (UV-6300PC, VWR) using a calibration curve of trastuzumab or IgG-B12.

### HPLC

Analytical high-performance liquid chromatography (HPLC) analyses of DFO-Lx, AF-Lx and AF-Mal was performed using a Jasco HPLC system equipped with an ALLTIMA® C18 5μ column (4.6×250 mm) and linear gradients of MeCN/water, 0.1% TFA at a flow rate of 1 mL/minute.

HPLC analyses of the ADCs were performed using a Jasco HPLC system equipped with a SEPAX ZENIX®-C SEC-300 column (300 Å, 7.8×300 m) and SEPAX ZENIX®-C SEC-300 guard column (Sepax Technologies Inc., Newark, Del., USA) using a mixture of 0.05 mol/L sodium phosphate, 0.15 mol/L sodium chloride (pH 6.8), and 0.01 mol/L NaN₃, as the eluent at a flow rate of 1 mL/minute. The radioactivity of the eluate was monitored using an inline NaI(Tl) radiodetector (Raytest Sockett).

iTLC

Instant thin layer chromatography (iTLC) analysis of the ADCs was carried out to assess the radiochemical purity of the ADC. Silica-impregnated glass fiber sheets (PI Medical Diagnostic Equipment BV) were used with 20 mmol/L citrate buffer (pH 5.0)/MeOH (3/7) as the mobile phase. As a readout gamma-well isotope counting with a gamma-well counter (Wallac LKB-CompuGamma 1282; Pharmacia) for ⁸⁹Zr was used.

### SDS-PAGE

The structural integrity of the ADC was determined with SDS-PAGE. Samples were mixed at 1:1 with loading buffer and run on a PHASTGEL® System (GE Healthcare Life Sciences) using preformed 7.5% SDS-PAGE gels under non-reducing and reducing conditions. The gel was analyzed by isotope counting using a phosphor imager and quantified with ImageQuant™ software.

### Binding Assay

Binding characteristics of the ADCs were determined in an immunoreactivity assay essentially as described by Lindmo et al. (28)[“1984, Lindmo”] using a serial dilution of 0.2% glutaraldehyde-fixed SKOV cells and a fixed amount of ⁸⁹Zr-DFO-Lx-trastuzumab or ⁸⁹Zr-DFO-trastuzumab. After overnight incubation at 4° C., the cell suspension was centrifuged and the specific binding calculated as the ratio of cell-bound radioactivity to the total amount of radioactivity in the assay. This was corrected for nonspecific binding, as determined with a 500-fold excess of nonradioactive trastuzumab. All binding assays were performed in triplicate.

### LC-MS

LC-MS analyses were performed using a Zenix LC system (Thermo Finnigan, San Jose, Calif., USA) coupled to a Bruker Q-TOF mass spectrometer (Bremen, Germany) equipped with an electrospray ionization (ESI) source. Mass determination was performed using a ZENIX®-C column (4.6×300 mm; 5 μm; Sepax Technologies Inc., Newark, Del., USA). The mobile phase consisted out of a mixture of water, acetonitrile, trifluoroacetic acid and formic acid (79.9/19.9/0.1/0.1, v/v/v/v, respectively). A 17-minute isocratic run was performed at a flow rate of 350 μL/minute. 10 μL of sample was injected. The LC flow was directed to the MS source from 2 to 10 minutes using the switch valve present on the mass spectrometer. The rest of the solvent flow was directed to waste to prevent source contamination. MS analysis was done in positive ionization mode using the following settings: ESI voltage, 4.5 kV; dry gas temperature, 190° C.; dry gas flow rate, 8 L/minute; nebulizer pressure, 1.6 bar; in-source collision-induced dissociation energy, 120 eV; ion energy, 5 eV; collision cell energy, 15 eV. Data was analyzed using Bruker Daltonics Data Analysis software. Protein ion charge assignment and molecular mass determinations were performed using the “Charge Deconvolution” utility of the Data Analysis software.

### Free Drug Determination

The percentage of free drug in the ADC solution was determined by precipitating 50 μL ADC with 100 μL acetonitrile, followed by centrifugation (10,000 rpm, 5 minutes) and C18 reversed-phase HPLC analysis of the supernatant. For quantification, a calibration curve was prepared using an AF-Lx-thiourea complex.

### Molecular Characterization of Lx-Based ADCs

Determination of the payload (i.e., functional moiety) position in the mAb was carried out by DTT reduction, pepsin or papain digestion of the ADC followed by HPLC, SDS-PAGE or SEC-MS analysis. ADCs (1 mg/mL in PBS) were deglycosylated with PNGase F (Sigma Aldrich) using 2 units/μg of enzyme per 100 μg antibody. The samples were incubated at 37° C. for 24 hours. Reduction of the cysteine bridges of the ADC was carried out by incubating ADCs (100 μL, 2 mg/mL in PBS) with DTT (100 μL, 100 mM in H₂O) for 20 minutes at 65° C. Pepsin digestions were carried out by rebuffering and concentrating the ADCs to 1 mg/mL in a NaOAc buffer (20 mM, pH 4.0) and subsequently, pepsin (Sigma Aldrich) (2.5 μL, 1 mg/mL in 10 mM HCl) was added. The mixture was incubated for 24 hours at 37° C., after which the pepsin was deactivated by adding Tris buffer (10 μL, 2 M, pH 8.0). Papain digestions were carried out by rebuffering and concentrating the ADCs to 1 mg/mL in a Tris-HCL buffer (100 mM, pH 7.6 containing 2 mM EDTA and 5 mM cysteine) and subsequently, papain (Sigma Aldrich) (4 μL, 0.5 mg/mL in H₂O) was added. The mixture was incubated for 4 hours at 37° C.

### Quality Tests and Stability of Lx-Based ADCs

The ADCs ⁸⁹Zr-DFO-Lx-trastuzumab and ⁸⁹Zr-DFO-trastuzumab were incubated with 1 volume equiv. 0.9% NaCl or 50% human serum at 37° C. At different time points, radiochemical purity of the conjugates was measured by iTLC and radio-HPLC, while conjugate integrity was analyzed by SDS-PAGE followed by phosphor imager analysis. In vitro binding characteristics of the ADCs were determined with the Lindmo binding assay.

### In Vitro Cell Viability Assay

The effects of the AF-Lx, AF-Mal, AF-Lx-trastuzumab, AF-Lx-IgG-B12, AF-Mal-trastuzumab, AF-Mal-IgG-B12, T-DM1 and trastuzumab on cell viability of the cell lines MDA-MB231, JIMT, BT-474, SKBR3, SKOV-3, and NCI-N87 were measured with the CELLTITER BLUE® Assay (Promega). Before starting the assay, the AF-Lx and AF-Mal were quenched by incubation the compounds with 1 mol equiv. thiourea (2 hours, 37° C.) and n-acetyl-cysteine (30 minutes, 0° C.), respectively. The cells were trypsinized and plated in 96-well, flat-bottomed, tissue culture plates at day 0. On day 1, from the above mentioned compounds serial dilution series (9×4-fold dilutions) were made starting from 10 or 1000 nM (depending on type of cells and compounds) and added to the cells (in case of ADCs, concentration of the mAb was used). On day 5, the CELLTITER BLUE® reagent was added and incubated for 2 hours at 37° C., and viable cells were measured with a TeCan plate reader (Tecan Group Ltd.) at 560EX/590EM nm. Fluorescence values of the samples were corrected for background of cell culture medium, the results, presented as the percentage of survival, were calculated by dividing the fluorescence values of the treated cells by the values of the untreated control cells. The data were analyzed with Graphpad Prism 5 software.

### Biodistribution of Lx-Based ADCs

Biodistribution of ⁸⁹Zr-DFO-Lx-trastuzumab, ⁸⁹Zr-DFO-trastuzumab, AF-Mal-trastuzumab-⁸⁹Zr and AF-Lx-trastuzumab-⁸⁹Zr conjugates was compared in female nude mice (Hsd athymic nu/nu, 25-32 g; Harlan CPB) either in non-tumor-bearing mice or mice bearing NCI-N87 tumors on both flanks. All animal experiments were performed according to the Dutch National Institutes of Health principles of laboratory animal care and Dutch national law (“Wet op de proefdieren,” Stb 1985, 336). Mice were injected (i.v.) a total volume of 100 μL with 2.0 MBq (100 μg mAb). Blood was collected by tail laceration at 1, 4, 24, 48, 72, and 96 hours p.i. At 72 or 96 hours after injection, the mice were anesthetized, bled, euthanized, and dissected. Blood and organs were weighed, and further processed. The amount of radioactivity in each sample was measured in a gamma counter. Radioactivity uptake was calculated as the percentage of the injected dose per gram of tissue (% ID/g).

In Vivo MTD and Therapy Study with Lx-Based ADC

Before starting the therapy study, the MTD for AF-Lx-trastuzumab and AF-Mal-trastuzumab was determined. For this purpose, five groups of 5 nude mice were given 15, 30, or 60 mg/kg AF-Lx-Trastuzumab, AF-Mal-Trastuzumab or normal saline as a control by an i.p. bolus injection. Body weight was measured three times per week and MTD was reached when weight loss was >10% compared with the control mice. The therapeutic effectiveness of AF-Lx-trastuzumab, AF-Mal-trastuzumab and T-DM1 was studied in the same nude mice models as described for the biodistribution study. For this purpose, seven groups of 8 or 9 mice with established NCI-N87 or JIMT-1 xenografts in both flanks were used. The mean tumor size at the start of the study was 140±30 mm³ and 140±30 mm³ for the study with the NCI-N87 tumors or the JIMT-1 tumors, respectively, and was similar for the different treatment groups. All mice received an i.p. bolus injection. In the study with the NCI-N87 tumors, Group 1 was the control group and received 100 μL of saline solution. Group 2 received 15 mg/kg trastuzumab, Group 3 received 15 mg/kg ado-trastuzumab emtansine (T-DM1), group 4 received 15 mg/kg AF-Lx-IgG-B12, Group 5 received 15 mg/kg AF-Mal-trastuzumab and Group 6 and 7 received 15 mg/kg and 5 mg/kg of AF-Lx-trastuzumab, respectively. Ado-trastuzumab emtansine was included in this study as a reference ADC with proven clinical efficacy. AF-Lx-IgG-B12 was included as a non-binding control and AF-Mal-trastuzumab was added as a benchmark conjugate. In the study with the JIMT-1 tumors, Group 1 was the control group and received 100 μL of saline solution. Group 2 received 15 mg/kg trastuzumab, Group 3 received 15 mg/kg ado-trastuzumab emtansine, Group 4 and 6 received 15 and 30 mg/kg AF-Lx-trastuzumab, respectively, and Group 5 and 7 received 15 mg/kg and 30 mg/kg AF-Mal-trastuzumab, respectively. Body weight and tumor volume were measured three times per week up to 4 months after end of treatment.

### Statistical Analysis

All animal experiments were statistically analyzed using the Welch's t test for independent samples. Two-sided significance levels were calculated, and P<0.05 was considered statistically significant.

### Results

Synthesis of Desferrioxamine-Lx Complexes (DFO-Lx), their Conjugation to mAbs, and Labeling with ⁸⁹Zr

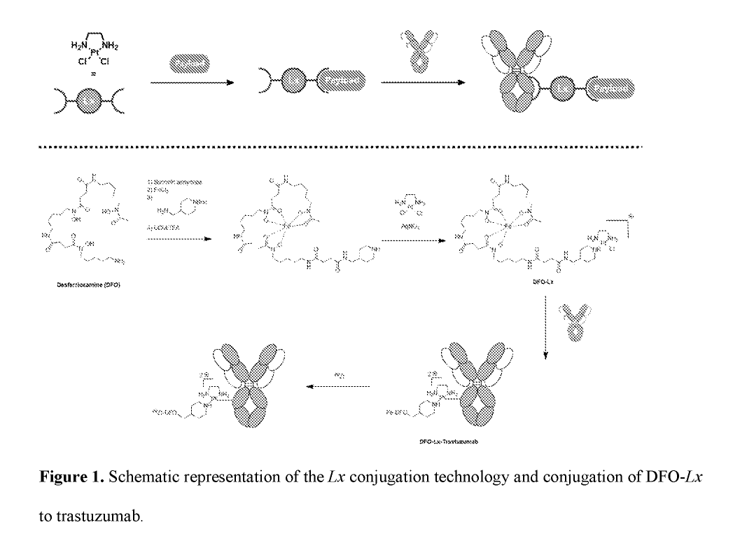

The method used in this disclosure for coupling the functional moieties to targeting moieties (also referred to as the Lx technology) is a two-step method for the conjugation of (toxic) payloads (functional moieties) to proteinaceous carriers (targeting moieties) such as monoclonal antibodies (FIG. 1). In the first step, the functional moiety is coordinated to the linker, i.e., cis-platin derivative [Pt(en)Cl₂] (i.e., “Lx linker” or “Lx”) followed by conjugation of the payload-Lx complex to the targeting moiety, e.g., a native mAb.

Although, desferrioxamine (DFO) could be directly coordinated to Lx linker via the primary amine, it was decided to first modify the DFO with a succinic acid group followed by the addition of a piperidine coordination group (FIG. 1). Coordination to Lx linker was effected by activation of [Pt(en)Cl₂] with AgNO₃ followed by reaction with the piperidine modified DFO having iron (Fe(III)) chelated. Purification of the crude product was performed by preparative HPLC to afford the DFO-Lx complex in >95% purity (λ 430 nm). The complex was stored as a 5 mM solution in water containing 20 mM NaCl with no decomposition observed by HPLC upon storage for at least 1 year at 4° C.

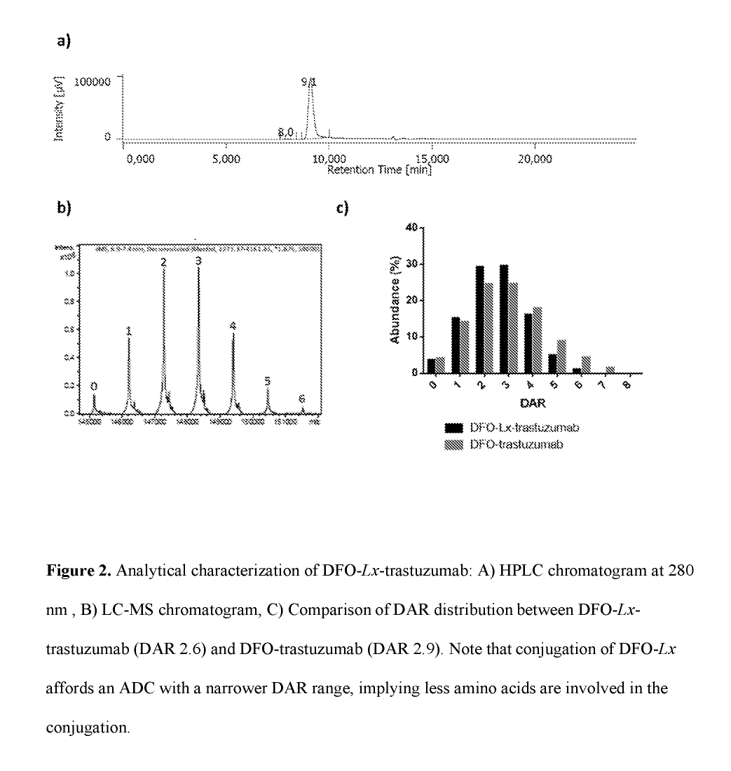

Conjugation of the Lx complex (i.e., functional moiety-linker complex) to mAbs was carried out in a straightforward way, the DFO-Lx complex was incubated with trastuzumab for 24 hours at 37° C. (FIG. 1). After purification, conjugation was clearly observed with size exclusion chromatography (SEC) and no aggregation was observed (FIG. 2, Panel a)). The DFO-Lx:mAb ratio, from now on referred to as functional moiety to targeting moiety ratio or Drug-Antibody-Ratio (DAR), was readily determined by mass spectrometry and was 2.6 (FIG. 2, Panel b)).

The versatility of the conjugation method was tested by conjugating trastuzumab, obinutuzumab and ofatumumab with the DFO-Lx complex under identical conjugation conditions. The mAb solutions were buffer-exchanged before conjugation in a 20 mM tricine buffer (pH 8.5) to 21 mg/mL affording conjugates (99% monomeric) with average DARs between 2.9 and 3.2 as determined with LC-MS (FIG. S1). A consistent distribution profile was observed. The percentage of unconjugated antibody was very low (1-5%), while conjugates with a DAR in the range of 2-4 and 1-5 represented 76-78%, respectively, 94-96% of the molecules.

In the biodistribution studies, DFO-Lx-trastuzumab was benchmarked against a lysine conjugated analogue (DFO-trastuzumab). The activated ester of the succinylated DFO was conjugated to the lysine residues in the mAb affording a conjugate with an average DAR of 2.9 [Verel et al REF]. Interestingly, a narrower distribution profile was found for DFO-Lx-trastuzumab compared to DFO-trastuzumab, suggesting that a smaller number of amino acid sites is involved in the coupling to DFO-Lx (FIG. 2, Panel c)).

Radiolabeling of DFO-based conjugates with ⁸⁹Zr was carried out according a procedure described by Verel et al. (26) [“2003, Verel”], implying removal of Fe(III) by trans-chelation to ethylenediamine tetraacetic acid (EDTA) and labelling with ⁸⁹Zr. The radiochemical purity of both conjugates was >99% and the immunoreactive fraction was similar for ⁸⁹Zr-DFO-Lx-trastuzumab and ⁸⁹Zr-DFO-trastuzumab. Moreover, SDS-PAGE and HPLC analyses showed a monomeric product, which indicates that the structural integrity of the mAb remained preserved upon conjugation and radiolabeling.

Synthesis of AF-Lx Complexes and their Conjugation to mAbs

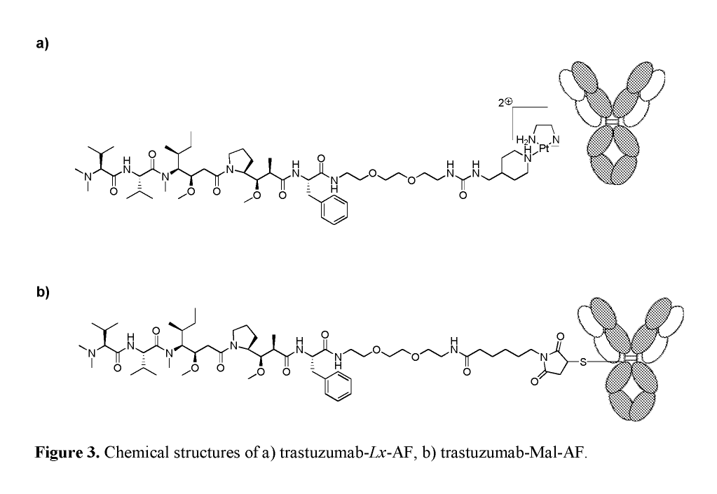

The highly potent Auristatin F (AF) was chosen as the cytotoxic payload for evaluation of the performance of Lx linker in therapeutic ADC approaches. It has been shown that the carboxylic group can be modified with a non-cleavable spacer without hampering its activity (29, 30) [“2006, Doronina” and “2012, Axup”]. To allow stable coupling to the Lx linker, AF was modified with a piperidine coordination group (FIG. 3, Panel a)). An ethylene glycol spacer between the drug and linker was added to improve the water solubility of the drug, which is further improved by the positive charge of the platinum.

The coordination of the piperidine modified AF with the Lx linker and subsequent conjugation of the formed AF-Lx complex to mAbs was similar as described for the DFO system. Conjugations of AF-Lx to trastuzumab afforded ADCs that were >99% monomeric with DARs in a range between 2.5 and 2.7.

To evaluate whether the Lx linker modifies the therapeutic efficacy of AF-based ADCs (FIG. 3, Panel a)), a drug-linker benchmark was designed having a maleimide conjugation group instead of an Lx linker (FIG. 3, Panel b)).

Maleimide chemistry is the conventional conjugation method to conjugate auristatin-based payloads to mAbs, like was performed in conjugating MMAE to brentuximab affording the FDA approved ADC ADCETRIS®. The maleimide moiety reacts with free cysteine thiols obtained via reduction of the cysteine bridges in the hinge region of the mAb. In order to prepare conjugates having a DAR comparable to the Lx-based ADC, the mAbs were partially reduced as described in the Materials and Methods section. The conjugation method afforded AF-Mal-trastuzumab ADCs that were >99% monomeric and having average DARs ranging from 2.0 to 2.3.

### Molecular Characterization of Lx-Based ADCs

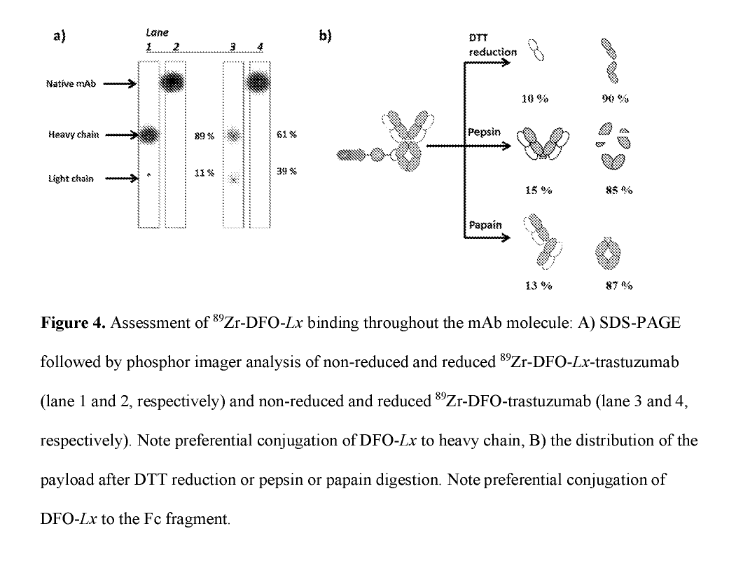

To assess the position of the Lx conjugated payload in the antibody, the ⁸⁹Zr radio-labeled conjugates were reduced with DTT to separate the heavy chain (HC) from the light chain (LC). SDS-PAGE followed by phosphor imager analysis of ⁸⁹Zr-DFO-Lx-trastuzumab showed preferential binding to the HC (89% of radioactivity bound to HC, 11% to LC) whereas for ⁸⁹Zr-DFO-trastuzumab a HC/LC ratio of 61:39 was found (FIG. 4, Panel a)). SEC-MS analysis of the Fe-labeled antibody conjugates afforded identical HC/LC distributions as found for the ⁸⁹Zr-labeled counterparts (FIG. S2, Panels a-b). Next to this, also SEC-MS analysis of reduced AF-Lx-trastuzumab confirmed the preferential conjugation of the Lx linker to HC.

To obtain further insight in the coupling position of the Lx-payload (i.e., functional moiety) in the mAb, AF-Lx-trastuzumab was digested with pepsin or papain in order to separate the Fc region from the F(ab)₂ or Fab, respectively (FIG. 4, Panel b) or FIG. S3). SEC-MS analysis on the pepsin digested AF-Lx-trastuzumab revealed an average DAR on the F(ab)₂ portion of 0.38, which is 15% of the total payload (DAR 2.7). In other words, ca. 85% of the payload is located in the Fc part of the mAb. This site restricted conjugation was confirmed by SEC-MS analysis on the papain digested AF-Lx-trastuzumab. Moreover, similar payload position distributions were found for a variety of payload-Lx and mAb combinations.

### Quality Tests and Stability of Lx-Based ADCs

The in vitro serum stability of the Lx-based ADCs was determined at 37° C. in 50% human serum with ⁸⁹Zr-DFO-Lx-trastuzumab, because the use of such radioimmunoconjugate as a model for testing of Lx performance in vitro and in vivo allows accurate and easy quantification (FIG. S4). Radio-TLC, Radio-HPLC and SDS-PAGE followed by phosphor imager analysis showed no release of ⁸⁹Zr upon incubation for up to 200 hours. Under these conditions the immunoreactive fraction, as determined by Lindmo binding assay, decreased with 3-4% after ˜300 hours. Results of these quality tests were very similar for ⁸⁹Zr-DFO-Lx-trastuzumab and ⁸⁹Zr-DFO-trastuzumab, the latter representing a conjugate obtained by covalent coupling and evaluated in many preclinical and clinical ⁸⁹Zr-immuno-PET studies (FIG. S4, Panels a-b).

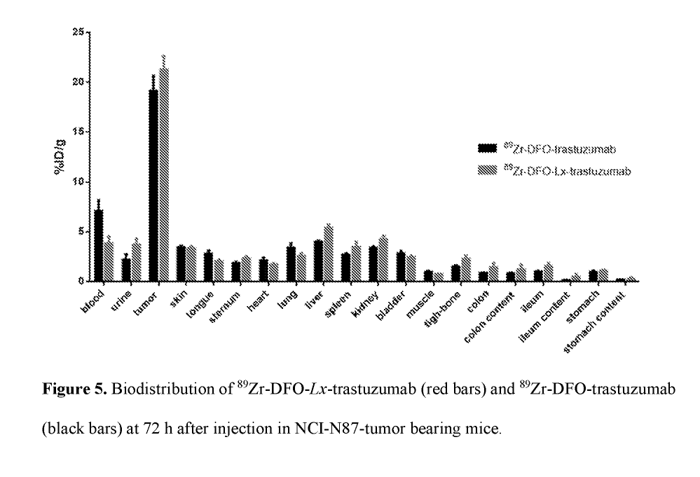

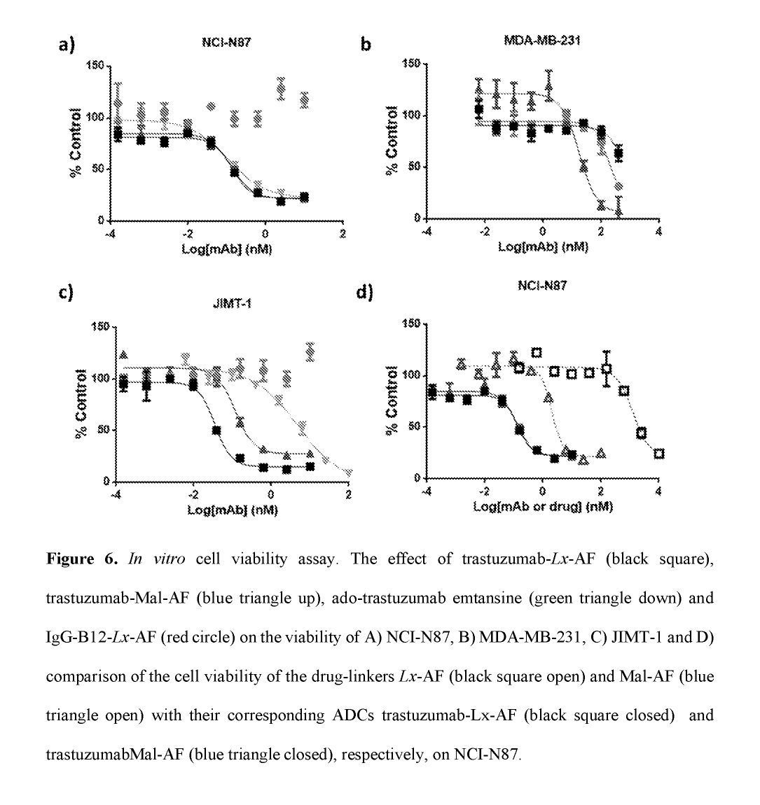

To assess the in vivo stability and tumor targeting potential of Lx-based ADCs, a biodistribution study was performed in nude mice bearing subcutaneous HER2-expressing NCI-N87 xenografts on both flanks (FIG. 5, Panel b)). ⁸⁹Zr-DFO-Lx-trastuzumab and ⁸⁹Zr-DFO-trastuzumab were prepared with a relatively low DFO:mAb ratio of 0.8 and 0.6, respectively, to avoid pharmacokinetic effects introduced by conjugation overload. Overall, ⁸⁹Zr-levels in organs were similar for both conjugates with the exception of liver (3.8±1.7 and 7.1±2.4) and blood (5.4±0.8 and 4.0±0.3 for ⁸⁹Zr-DFO-Lx-trastuzumab and ⁸⁹Zr-DFO-trastuzumab, respectively)

⁸⁹Zr-levels in tumor were similar for both conjugates, while with ⁸⁹Zr-DFO-Lx-trastuzumab higher tumor-to-blood ratios were obtained demonstrating that incorporation of the platinum(II)-linker allows for stable conjugation and efficient tumor targeting.

### In Vitro Cell Viability Assay

The cell killing potential of the cell targeting conjugate comprising auristatin as a functional moiety, trastuzumab as a targeting moiety and the platinum complex linker Lx (hereinafter AF-Lx-trastuzumab) was determined.

AF-Lx-trastuzumab was measured with the CELLTITER BLUE® assay and compared with its maleimide conjugated analogue, AF-Mal-trastuzumab, and ado-trastuzumab emtansine. Moreover, an Lx-based ADC comprising the non-tumor binding mAb IgG-B12, AF-Lx-IgG-B12, was included in the experiments to serve as a negative control.

AF-Lx-trastuzumab, AF-Mal-trastuzumab and ado-trastuzumab emtansine showed similar sub-nanomolar potencies in the HER2-positive cell lines NCI-N87, SKOV-3, SK-BR3 and BT-474 with IC₅₀'s between 10 and 200 pM (FIG. 6, Panel a), Table S1). On the other hand, the non-binding ADC, AF-Lx-IgG-B12, did not reveal an IC₅₀ in the measured range indicating that the cells are killed in an antigen dependent manner. This is supported by the low potencies of the ADCs in the HER2-negative cell line MDA-MB-231 (FIG. 6, Panel b)). The lower IC₅₀ of T-DM1 suggests that DM1 becomes released during the experiment. Interestingly, both AF-based ADCs showed sub-nanomolar potencies in the T-DM1-resistant JIMT-1 cell line whereas the IC₅₀ of T-DM-1, as would be expected, was an order of magnitude higher, while contribution of free DM1 cannot be excluded.

To anticipate what might happen when the drug-linkers AF-Lx and AF-Mal become freely available, also the toxicity of these compounds was determined in all tested cell lines (FIG. 6, Panel d)). Before the experiment, the AF-Lx and AF-Mal were quenched by incubating the compounds with thiourea and n-acetyl-cysteine, respectively. In all tested cell lines, the potency of the drug-Lx combination was always 10³- to 10⁴-fold lower compared to its corresponding ADC whereas for the maleimide-based linker and corresponding ADC this difference is only 10 to 100 times. Moreover, by comparing the two drug-linker combinations it appears that the IC₅₀s of AF-Lx is always 10² to 10³ fold higher than AF-Mal.

### Biodistribution of Lx-based ADCs

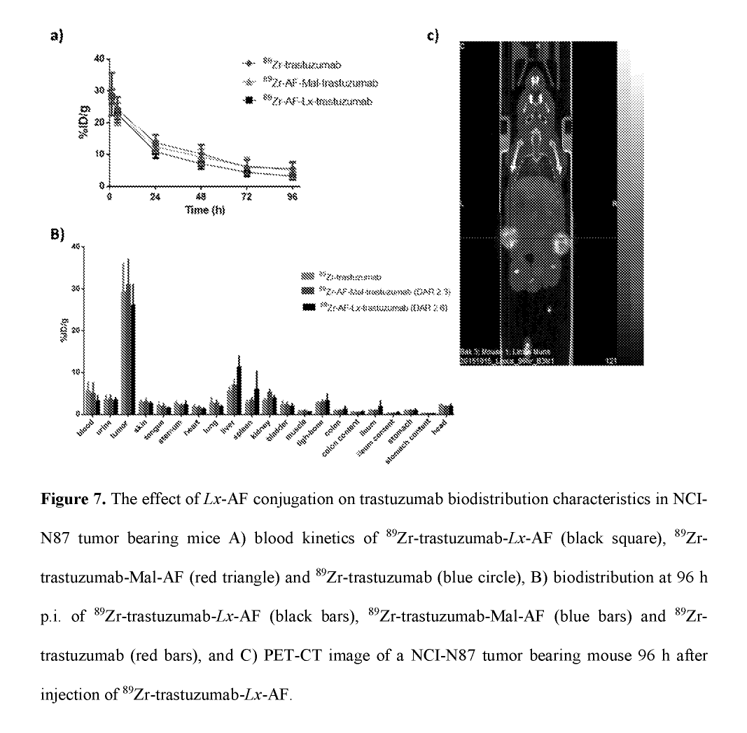

The effect of Lx-conjugated AF on mAb pharmacokinetics, tumor targeting and biodistribution was examined by injecting NCI-N87 tumor bearing mice with AF-Lx-trastuzumab-⁸⁹Zr, AF-Mal-trastuzumab-⁸⁹Zr or trastuzumab-⁸⁹Zr (FIG. 7).

The blood kinetics did not show significant differences: at 1 hour p.i. the blood levels were 28.2±2.2, 29.1±1.6 and 29.0±6.8 and slowly decreased to 3.2±1.2, 5.1±2.5 and 5.6±2.1 at 96 hours p.i. for trastuzumab-⁸⁹Zr, AF-Mal-trastuzumab-⁸⁹Zr and AF-Lx-trastuzumab-⁸⁹Zr, respectively (FIG. 7, Panel a)). The tumor uptake of the Lx-conjugated ADC (26.1±4.9) was similar to the uptake of the maleimide-conjugated ADC (31.0±6.0) and unconjugated trastuzumab (29.2±6.8). The excellent tumor selectivity of AF-Lx-trastuzumab-⁸⁹Zr is visualized by PET-CT in FIG. 7, Panel c). No significant differences in ⁸⁹Zr uptake were observed in normal tissues except for the liver, which was higher for AF-Lx-trastuzumab-⁸⁹Zr (11.6±2.4) than for AF-Mal-trastuzumab-⁸⁹Zr and trastuzumab-⁸⁹Zr (7.0±1.4 and 5.7±0.8, respectively).

In Vivo MTD and Therapy Study with Lx-Based ADC

An in vivo safety study was performed with AF-Lx-trastuzumab (DAR 2.3) and AF-Mal-trastuzumab (DAR 2.6). Non-tumor bearing mice were injected with 15, 30 or 60 mg/kg, single bolus injection. The mice treated with 30 mg/kg AF-Lx-trastuzumab showed a weight loss of 96% whereas the weight loss tended to increase above 10% for the mice treated with 60 mg/kg AF-Lx-trastuzumab. The weight loss of the mice treated with 60 mg/kg AF-Mal-trastuzumab was 96%.

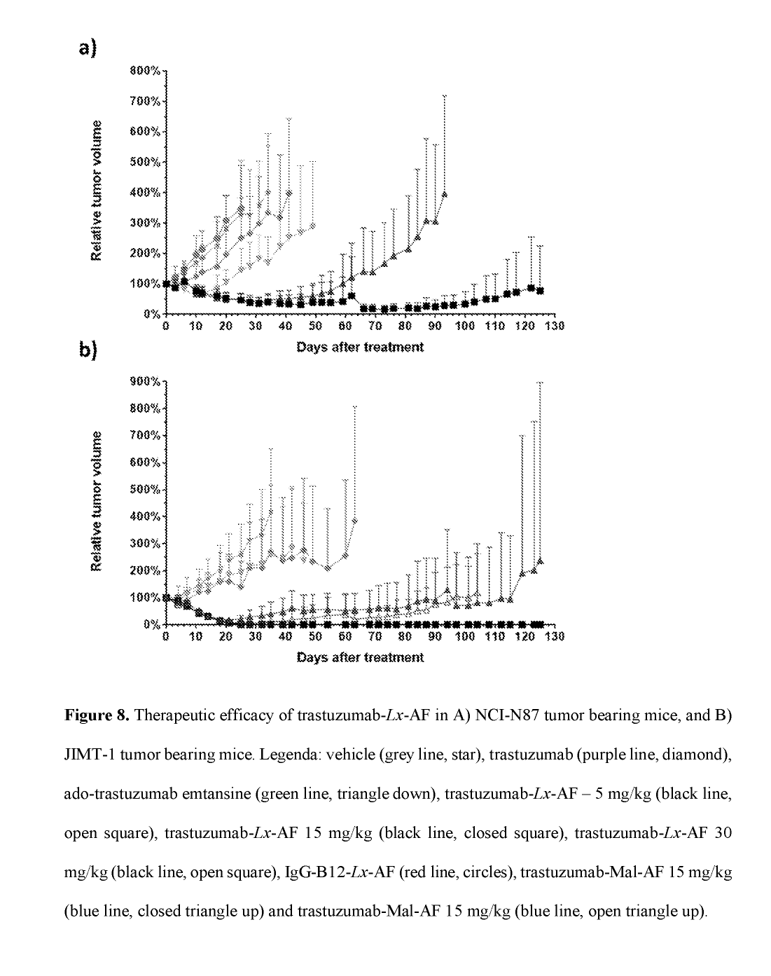

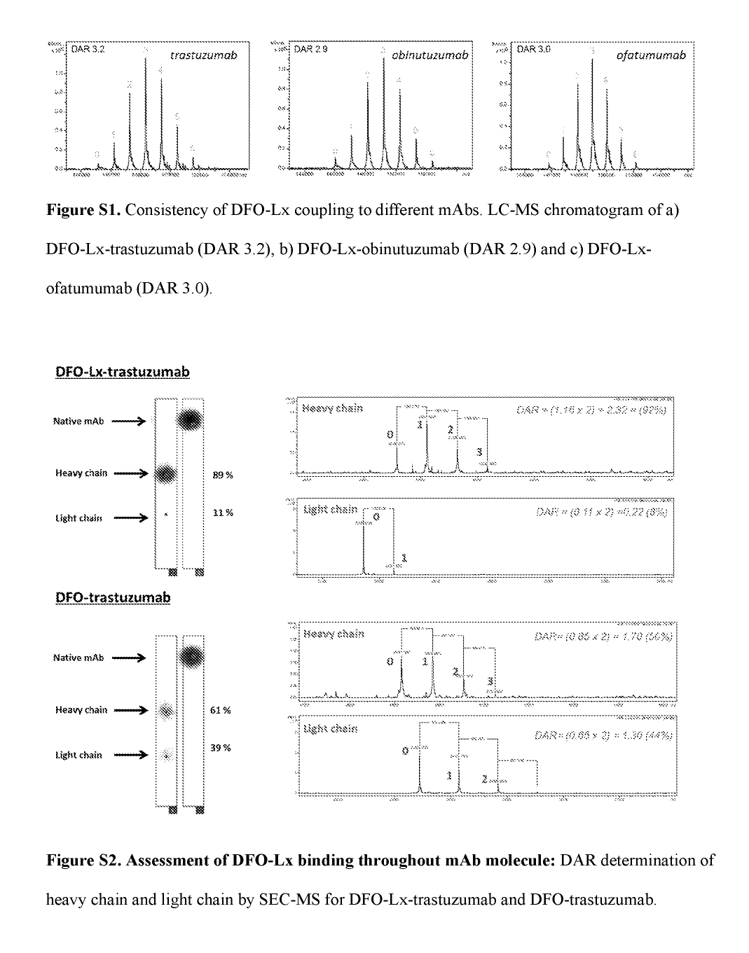

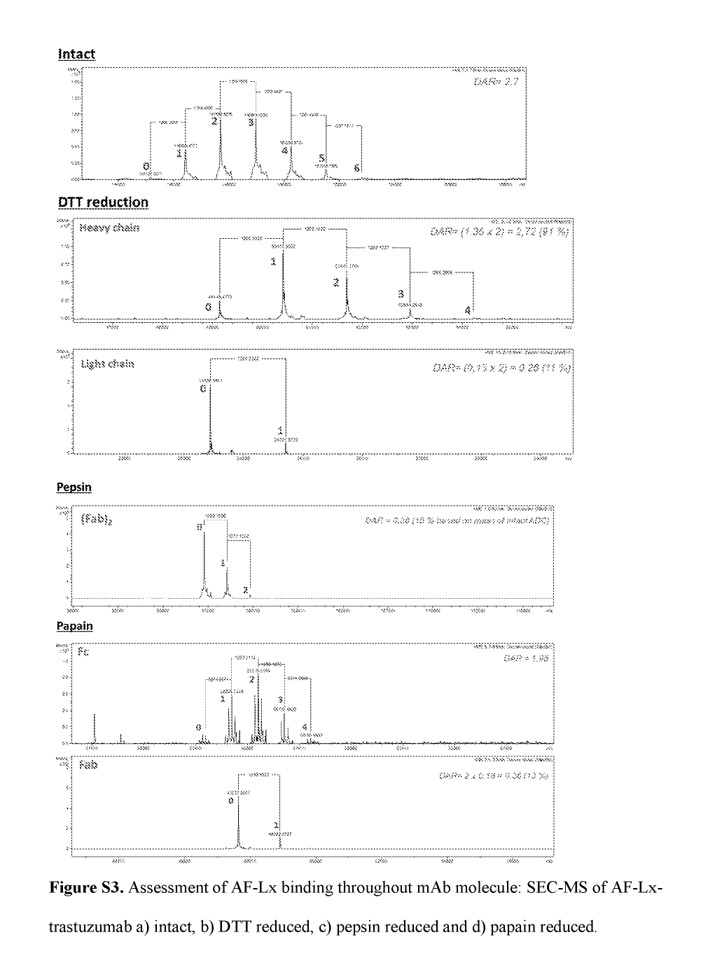

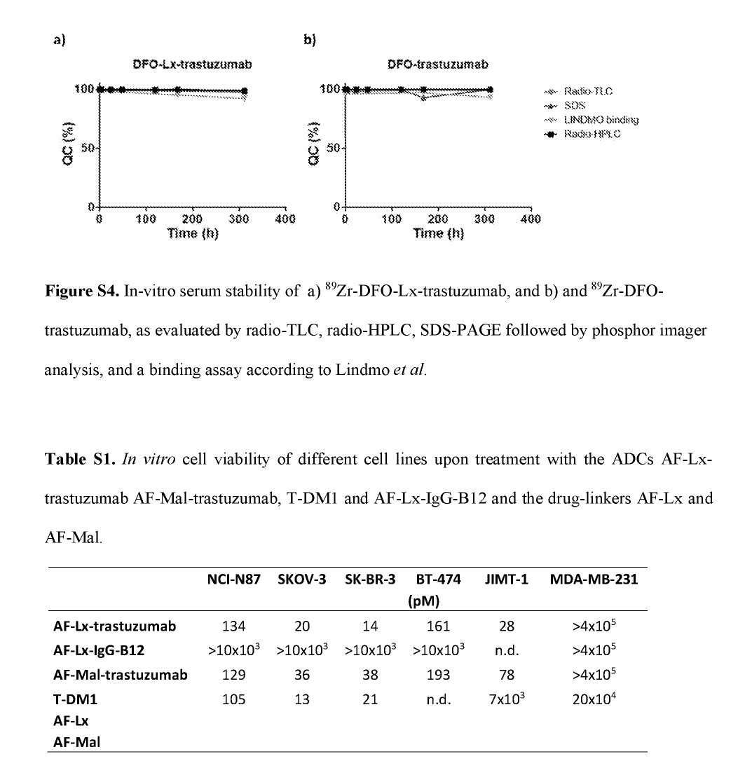

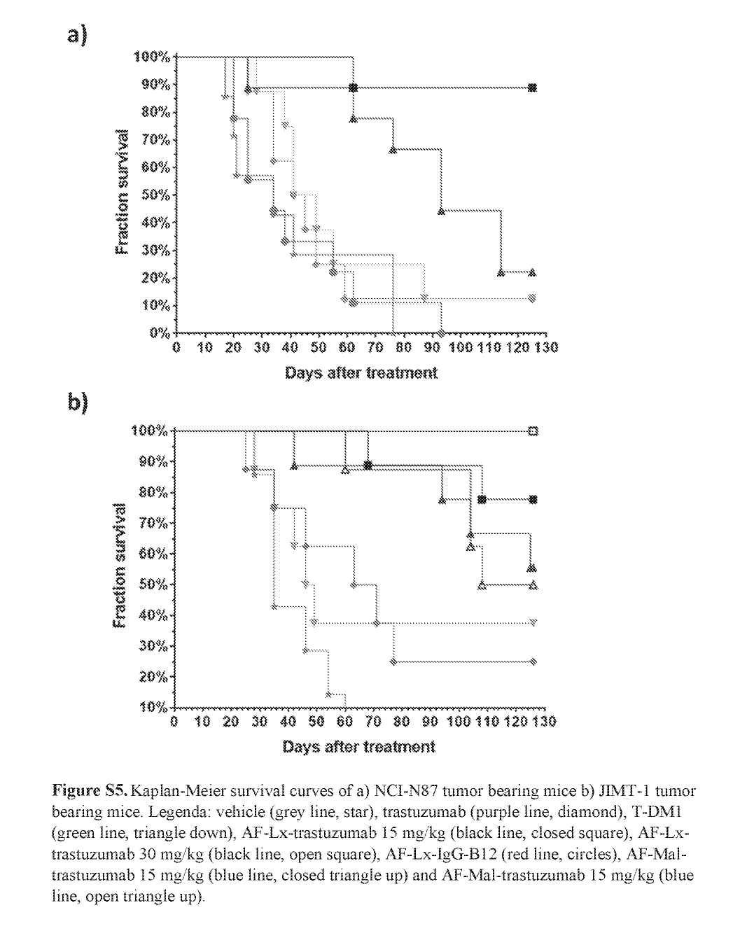

The in vivo efficacy of the AF-Lx-trastuzumab (DAR 2.6) was assessed in mice bearing NCI-N87 tumors (FIG. 8, Panel a)). Injections with PBS, trastuzumab and AF-Lx-IgG-B12 (DAR 2.4) were used as controls, while ado-trastuzumab emtansine and AF-Mal-trastuzumab (DAR 2.0) were included as benchmarks. All ADCs were administered as a single bolus injection at a dose of 15 mg/kg.

Initially, NCI-N87 tumors regressed after injected of trastuzumab ADCs whereas the non-binding control ADC trastuzumab caused only a delay in growth. While from day 20 on the tumors treated with ado-trastuzumab emtansine started regrowth, the tumors of both AF-based ADC groups continued regressing. Tumor growth differences between groups treated with AF-Lx-trastuzumab and AF-Mal-trastuzumab became clearly apparent after 60 days. Where the tumors of mice treated with AF-Mal-trastuzumab started regrowth at this time point, the tumors of mice treated with AF-Lx-trastuzumab remained constant in mean volume up to end of the experiment at day 125. Finally, 8 of the 9 mice treated with AF-Lx-trastuzumab survived the study whereas 2 of the 9 mice treated with AF-Mal-trastuzumab survived (FIG. S5, Panel a)). All mice dropped out of the study as a result of the tumor size (>1000 mm³). Cures, defined as no outgrowth of regressed individual tumors during the follow-up period, were observed for 12 of the 16 tumors (6 of the 9 mice) in the AF-Lx-trastuzumab treated group at day 125. The number of cures in the AF-Mal-trastuzumab treated group was 2 out of the 4 tumors.

The in vivo therapeutic efficacy of AF-Lx-trastuzumab was further evaluated in JIMT-1 xenografted mice. Although the JIMT-1 cell line is positive for HER2 expression, it has been shown that JIMT-1 tumors are trastuzumab as well as ado-trastuzumab emtansine resistant (27, 31-33) [“2004, Tanner”; “2010, Koninki”; “2015, Lagonzo”; and “Barok, 2015”]. The set-up of this study was similar as the efficacy study with the NCI-N87 xenografts, the DAR of the AF-Lx-trastuzuman and AF-Mal-trastuzuman was 2.6 and 2.3, respectively. Moreover, in this study two groups treated with a single bolus injection at a dose of 30 mg/kg AF-Lx-trastuzumab and 30 mg/kg AF-Mal-trastuzumab were included. Both trastuzumab and ado-trastuzumab emtansine treatment caused minimal growth delay (FIG. 8, Panel b)). On the other hand, tumors of mice treated with both AF-based conjugates regressed after injection. Whereas around day 20 after injection some tumors in both the AF-Mal-trastuzumab treated group started to regrow, all tumors in the AF-Lx-trastuzumab groups regressed completely and no regrowth was observed up to end of the experiment at day 125. Finally, 7 of the 9 mice treated with 15 mg/kg AF-Lx-trastuzumab survived the study whereas 9 of the 9 mice treated with 30 mg/kg AF-Lx-trastuzumab survived (FIG. S5, Panel b)). The survival rate of the maleimide benchmark group was 5 out of 9 and 4 out 8 for the groups treated 15 mg/kg and 30 mg/kg AF-Mal-trastuzumab, respectively.

### Supporting Information

TABLE S1
In vitro cell viability of different cell lines upon treatment with the ADCs AF-Lx-
trastuzumab, AF-Mal-trastuzumab, ado-trastuzumab emtansine and AF-Lx-IgG-
B12 and the drug-linkers AF-Lx and AF-Mal.

| | NCI-N87 | | | | | MDA- |
|---|---|---|---|---|---|---|
| | (pM) | SKOV-3 | SK-BR-3 | BT-474 | JIMT-1 | MB-231 |
| AF-Lx- | 134 | 20 | 14 | 161 | 28 | >4 × 10⁵ |
| trastuzumab | | | | | | |
| AF-Lx-IgG-B12 | >10 × 10³ | >10 × 10³ | >10 × 10³ | >10 × 10³ | n.d. | >4 × 10⁵ |
| AF-Mal- | 129 | 36 | 38 | 193 | 78 | >4 × 10⁵ |
| trastuzumab | | | | | | |
| T-DM1 | 105 | 13 | 21 | n.d. | 7 × 10³ | 20 × 10⁴ |
| AF-Lx | | | | | | |
| AF-Mal | | | | | | |

### Supporting Info Chemistry

### Synthesis of DFO- and AF-Lx Complexes and Corresponding Benchmarks

All reagents were used as purchased unless otherwise stated. ¹H and ¹³C nuclear magnetic resonance (NMR) spectra were recorded on a Bruker Avance™ 250 MHz, a Bruker MSL 400 MHz, a Bruker 500 MHz spectrometer where spectra were recorded at a temperature of 25° C. Chemical shifts (δ) are given in ppm and were internally referenced to residual solvent resonances (¹H: δ=7.29 ppm, ¹³C: δ=77.0 ppm). Flash column chromatography was performed with Aldrich silica gel (60 Å, 230-400 mesh). High resolution mass spectra (HRMS, ESI) were recorded on am Agilent mass spectrometer using ESI-TOF (Electrospray ionization-time of flight). Thin-layer chromatography (TLC) was performed on Merck silica plates (60E-254) and compounds were visualized by short-wavelength UV-light and KMnO₄ staining. Preparative-HPLC was performed using an ALLTIMA® C18 5μ column (22×250 mm) and linear gradients of water+0.1% TFA (eluent A) and MeCN+0.1% TFA (eluent B) at a flow rate of 10 mL/minute unless stated otherwise. Analytical high-performance liquid chromatography (HPLC) analyses was performed using a Jasco HPLC system equipped with an ALLTIMA® C18 5μ column (4.6×250 mm) and linear gradients of MeCN/water, 0.1% TFA at a flow rate of 1 mL/minute.

### Synthesis of Platinum Complex DFO-Lx

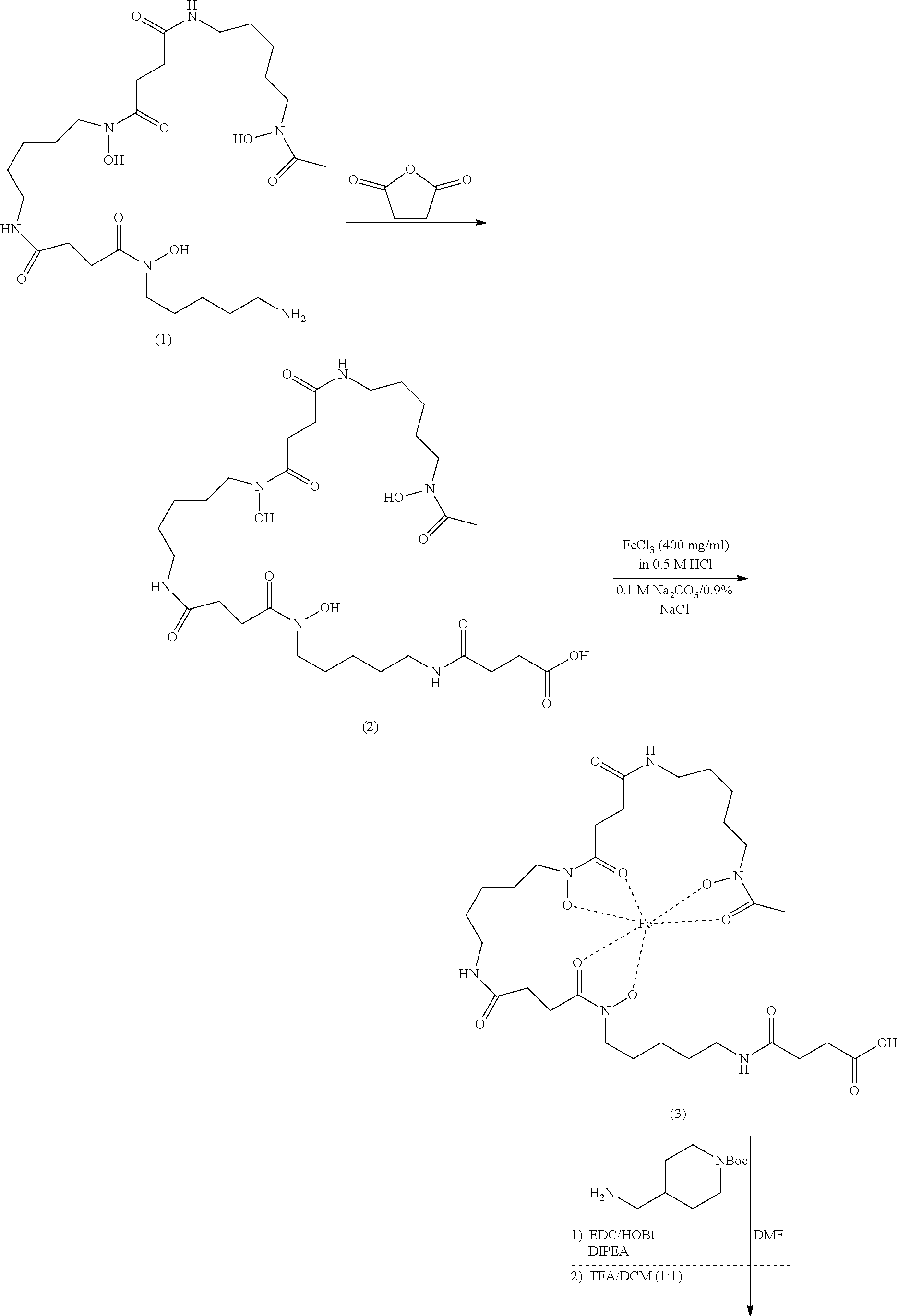

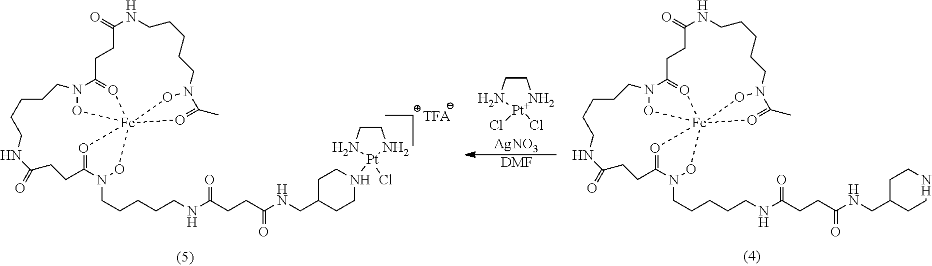

### N-Succinyl Desferrioxamine (1)

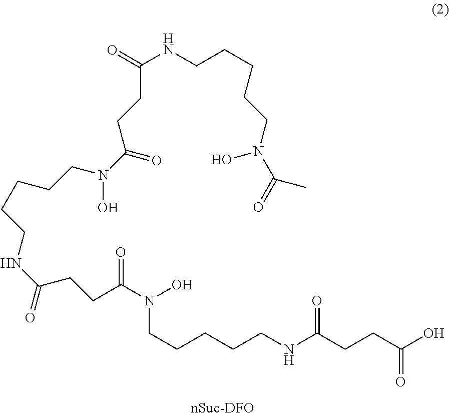 [SMILES: CC(=O)N(O)CCCCCNC(=O)CCC(=O)N(O)CCCCCNC(=O)CCC(=O)N(O)CCCCCNC(=O)CCC(=O)O]

N-succinyl desferrioxamine (N-suc-DFO) was prepared according to a procedure described by Verel et al. (I. Verel, G. W. M. Visser, R. Boellaard, M. Stigter-van Walsum, G. B. Snow, and G. A. M. S. van Dongen (2003), ⁸⁹Zr immuno-PET: Comprehensive procedures for the production of 89Zr-labeled monoclonal antibodies, J. Nucl. Med. 44, 1271-1281).

### Fe—N-Succinyl Desferrioxamine (3)

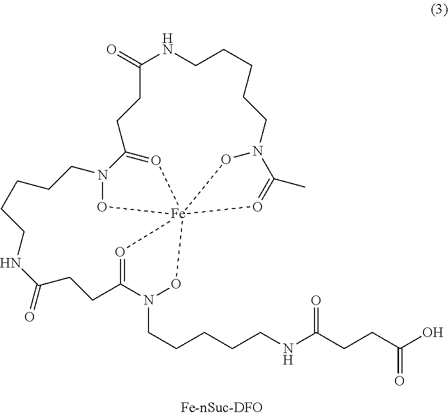

A stock solution of FeCl₃ (167 μL of 400 mg/mL in 0.5 M HCl) was added dropwise to a stirring solution of (2) (250 mg, 0.378 mmol) in 0.1 M Na₂CO₃ (5.5 mL) and 0.9% NaCl (4.8 mL). The mixture was stirred for 1 hour at RT after which a solution of 2.85% TFA in water (6 mL, 4 eq. trifluoroacetic acid (TFA) compared to DFO) was added and the volume was adjusted to ca. 30 mL with water. The solution was loaded on six SEP-PAK® C18 Plus cartridges (SEP-PAK® C18 Plus Short Cartridge, 360 mg Sorbent per Cartridge, 55-105 μm, Waters), 2 cartridges in series, which were activated with methanol (10 ml/cartridge) followed by water (60 ml/cartridge). The whole set up was then washed with water (100 ml/cartridge) after which the column was purged with air followed by elution of the product with acetonitrile (5 ml/cartridge). The product was diluted with an equal volume of water and lyophilized affording a brown solid (263 mg, 97%).

HPLC-analysis and mass spectrometry showed that the product was pure.

Fe—N-succinyl-(aminomethyl)piperidine desferrioxamine (4)

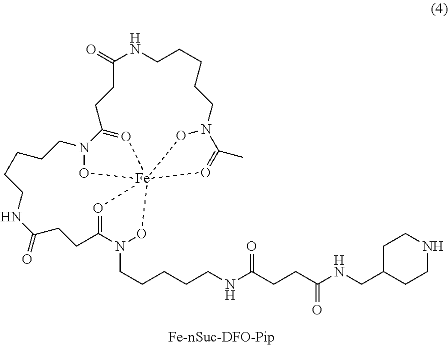

(3) (150 mg, 0.210 mmol) was dissolved in DMF (2.5 ml) and HOBt (42.6 mg, 0.315 mmol), EDC (60.4 mg, 0.315 mmol), DIPEA (0.036 ml, 0.315 mmol) and tert-butyl 4-(aminomethyl)piperidine-1-carboxylate (45 mg, 0.210 mmol) were sequentially added. The reaction was stirred for 20 hours and subsequently concentrated. The crude product was taken up in water/MeOH mixture (9/1, 10 mL) and loaded on 10 SEP-PAK® C18 plus columns (in parallel), which were activated with methanol (10 ml/cartridge) followed by water (60 ml/cartridge). The whole set up was then washed with water (100 ml/cartridge), after which the column was purged with air followed by elution of the product with acetonitrile (5 ml/cartridge). The product fraction was diluted with an equal volume of water and lyophilized to give 193 mg product. HPLC-analysis revealed the presence of HOBt.

HRMS (ESI+) C₄₀H₆₉FeN₈O₁₂ [M+H]⁺ calc. 910.4457, found 910.4464.

The lyophilized product was dissolved in 4 ml DCM/TFA (1:1) and stirred for 75 minutes at RT. Subsequently, the reaction mixture was concentrated, after which the solid was dissolved in 10 ml water and lyophilized. The product was dissolved in methanol and charged on one ISOLUTE® SCX-2 2G column (Biotage) that had been activated with methanol. The column was washed three times with 0.25 M ammonia in methanol and eluted with 15 ml 1 M ammonia in methanol and 40 ml 7 M ammonia in methanol, after which the solution was concentrated affording a brown solid (142 mg, 84%).

HPLC-analysis and mass spectrometry showed that the product was pure.

HRMS (ESI+) C₃₅H₆₁FeN₈O₁₀ [M+H]⁺ calc 810.3933, found 810.4602.

### Synthesis of the platinum complex [Pt(en)(Fe—N-succinyl-(aminomethyl)piperidine desferrioxamine)Cl](TFA) (5)

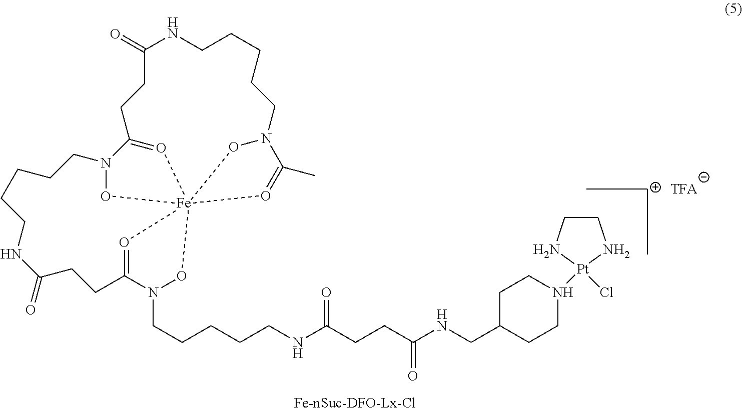

AgNO₃ (27.0 mg, 0.153 mmol) was added to a suspension of [Pt(en)Cl₂] (50 mg, 0.153 mmol) in DMF (1 mL) and stirred for 16 hours at RT in the dark. The mixture was filtered over Celite and the filter was rinsed with 1 ml DMF. Subsequently, 0.78 ml of the solution (0.062 mmol) was added to (4) (50 mg, 0.062 mmol) and the mixture was stirred for 16 hours at RT in the dark followed by removal of the solvent under reduced pressure. Purification by preparative-HPLC (10-25% MeCN in 40 minutes) followed by lyophilization of the product fractions afforded the product (25 mg, 37%) as a brown solid. HPLC-analysis and mass spectrometry showed that the product was pure. HRMS (ESI+) C₃₇H₆₉ClFeN₁₀O₁₀Pt [M+H]⁺ calc 1100.3951, found 1100.3694.

### Synthesis of the platinum complex Auristatin F-Lx (AF-Lx)

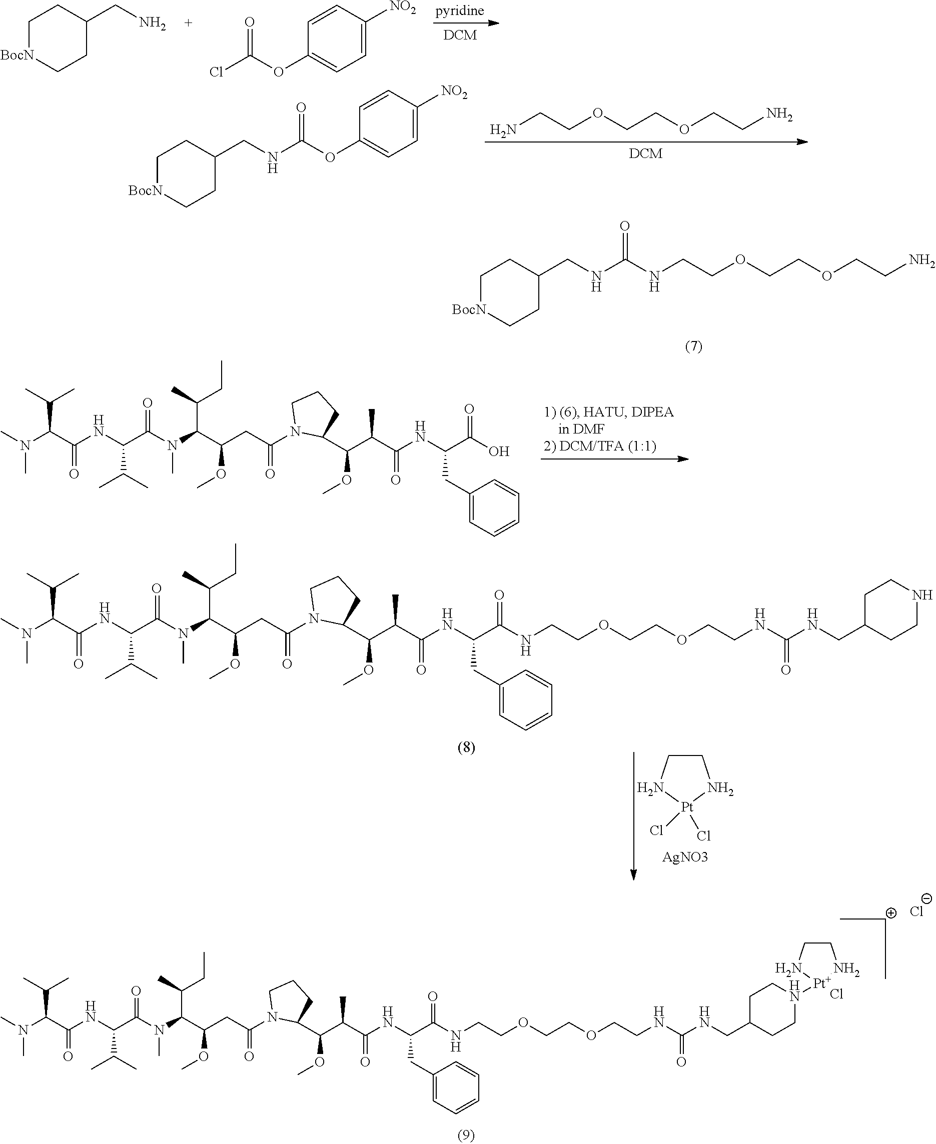

### tert-butyl 4-((((4-nitrophenoxy)carbonyl)amino)methyl)piperidine-1-carboxylate (6)

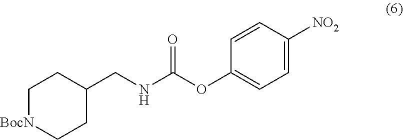 [SMILES: CC(C)(C)OC(=O)N1CCC(CNC(=O)Oc2ccc([N+](=O)[O-])cc2)CC1]

4-nitrophenyl carbonochloridate (2.573 g, 12.722 mmol) was added to a solution of tert-butyl 4-(aminomethyl)piperidine-1-carboxylate (2.737 g, 12.772 mmol) and pyridine (1.1 ml, 12.772 mmol) in DCM (66 ml) under argon at RT. The reaction was heated to reflux and stirred for 6 hours. Subsequently, the reaction was concentrated and washed with sat. aq. NaHCO₃ (2×), water and brine, dried with Na₂SO₄, filtered and concentrated. Purification by flash chromatography (10-50% EtOAc in cHex) afforded the product (6) (2.12 g, 47%) as a crème solid.

¹H-NMR (400 MHz, CDCl₃) δ 8.23 (d, J=9.1 Hz, 2H), 7.30 (d, J=9.1 Hz, 2H), 5.37 (t, J=5.9 Hz, 1H), 4.14 (d, J=13.5 Hz, 2H), 3.18 (t, J=6.1 Hz, 2H), 2.70 (t, J=12.2 Hz, 2H), 1.78-1.66 (m, 3H), 1.45 (s, 9H), 1.17 (qd, J=12.5, 3.7 Hz, 2H).

¹³C-NMR (125 MHz, CDCl₃) δ 155.8, 154.7, 153.3, 144.6, 125.1, 121.9, 79.5, 46.7, 43.4, 36.5, 29.5, 28.4.

HRMS (ESI⁺) C₁₈H₂₅N₃O₆[M+Na]⁺ calc 402.1636, found 402.1625.

### tert-butyl 4-(12-amino-3-oxo-7,10-dioxa-2,4-diazadodecyl)piperidine-1-carboxylate (7)

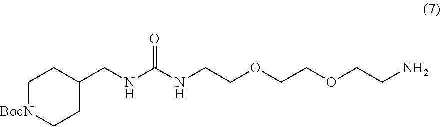 [SMILES: CC(C)(C)OC(=O)N1CCC(CNC(=O)NCCOCCOCCN)CC1]

To a stirring solution of triethylamine (0.11 ml, 0.790 mmol) and 2,2′-(ethane-1,2-diylbis(oxy))diethanamine (0.58 ml, 3.95 mmol) in DCM (10 ml) at RT, (5) (300 mg, 0.790 mmol) was added as a solution in DCM (5 mL). The reaction was stirred for 2 hours at RT. Subsequently, the reaction was diluted to 20 mL DCM and washed with 1 M NaOH (3×2 mL) and water (2×2 mL). The combined aqueous layers were extracted with DCM (10 mL), after which the combined organic layers were dried with Na₂SO₄, filtered and concentrated onto silica gel. Purification by flash chromatography (0-10% MeOH/NH₄OH (9:1) in DCM (fast) and then 10% MeOH/NH₄OH (9:1)+5% MeOH in DCM) afforded the product (7) (256 mg, 83%) as a pale yellow oil.

¹H-NMR (400 MHz, CDCl₃) δ 5.80 (t, J=5.9 Hz, 1H), 5.47 (t, J=5.5 Hz, 1H), 4.06 (br s, 2H), 3.62-3.57 (m, 4H), 3.55-3.52 (m, 4H), 3.35 (td, J=5.4, 4.7 Hz, 2H), 3.02 (br s, 2H), 2.89 (t, J=5.0 Hz, 2H), 2.64 (br t, J=11.7 Hz, 2H), 2.33 (s, 2H), 1.66-1.53 (m, 3H), 1.42 (s, 9H), 1.06 (qd, J=12.0, 4.4 Hz, 2H).

¹³C-NMR (125 MHz, CDCl3) δ 158.9, 154.8, 79.2, 72.1, 70.6, 69.9, 69.8, 45.5, 41.3, 40.0, 36.9, 29.7, 28.4.

HRMS (ESI+) C₁₈H₃₇N₄O₅ [M+H]⁺ calc 389.2759, found 389.2766.

### AF-(4-(12-amino-3-oxo-7,10-dioxa-2,4-diazadodecyl)piperidine) (8)

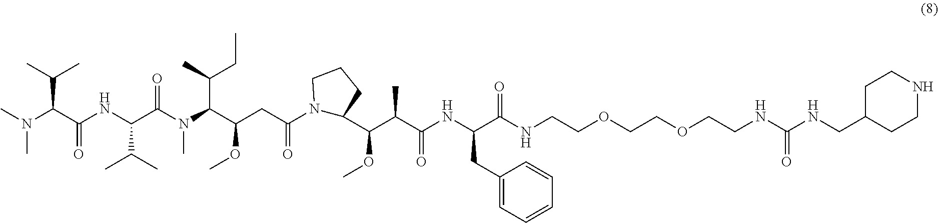 [SMILES: CC[C@H](C)[C@@H]([C@@H](CC(=O)N1CCC[C@H]1[C@H](OC)[C@@H](C)C(=O)N[C@H](Cc1ccccc1)C(=O)NCCOCCOCCNC(=O)NCC1CCNCC1)OC)N(C)C(=O)[C@@H](NC(=O)[C@H](C(C)C)N(C)C)C(C)C]

Auristatin F (AF, 29.8 mg, 0.040 mmol) (AF) dissolved in DMF (1 ml) was added to a solution of (7) (46.6 mg, 0.120 mmol) in DMF (1 ml). HATU (30.4 mg, 0.080 mmol) and DIPEA (0.021 ml, 0.120 mmol) were consecutively added and the mixture was stirred for 6 hours at RT, after which the reaction was concentrated. The product was taken up in 30% MeCN in water (4 ml) and purified by preparative-HPLC (30-50% MeCN in H₂O in 40 minutes) affording the product as a colorless solid.

HPLC-analysis and mass spectrometry showed that the product was pure.

HRMS (ESI+) C₅₈H₁₀₂N₉O₁₂ [M+H]+ calc 1116.7642, found 1116.7774.

The product was taken up in DCM (2 mL) and TFA (2 mL) was added, the mixture was stirred for 80 minutes at RT followed by concentration under reduced pressure. The product was taken up in 10% MeOH in DCM (2 ml) and loaded on an ISOLUTE® SCX-2 columns prewashed with DCM (10 ml). The column was washed with 10% MeOH in DCM (20 mL) and the product was eluted with 1 M methanolic ammonia in DCM (1:1). The combined product fractions were concentrated and co-evaporated with DCM several times to remove traces of ammonia affording the product (30.5 mg, 75%) as a colorless oil.

HPLC-analysis and mass spectrometry showed that the product was pure.

HRMS (ESI⁺) C₅₂H₉₁N₉O₁₀ [M+2H]²⁺ calc 508.8596, found 508.8642.

### Synthesis of platinum complex [Pt(en)(AF-(4-(12-amino-3-oxo-7,10-dioxa-2,4-diazadodecyl)piperidine))Cl](Cl) (9)

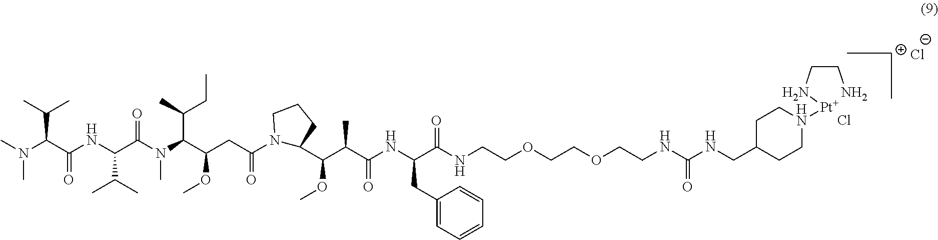

AgNO₃ (14.3 mg, 0.085 mmol) was dissolved in DMF (2 ml) and added to a suspension of [Pt(en)Cl₂] (50 mg, 0.153 mmol) in DMF (7.5 mL) and stirred for 24 hours at RT, after which the solution was filtered over celite. Subsequently, 3.52 ml of this solution (0.060 mmol) was added to a solution of (8) (30.5 mg, 0.030 mmol) in DMF (1 ml) and the mixture was stirred for 16 hours at RT in the dark, after which a 20 mM NaCl solution (2 ml) was added followed by the removal of the solvents under reduced pressure. The product was purified by preparative-HPLC (10-25% B in 40 minutes, eluent A: 20 mM NaCl in MILLI-Q®+0.1% TFA, eluent B: 9:1 MeCN: 20 mM NaCl in MILLI-Q®, +0.1% TFA) and the product fraction (ca. 20 mL) was concentrated to ca. 4 mL. Subsequently, the solution was diluted with 20 mM NaCl to ca. 20 mL and loaded on two SEP-PAK® C18 Plus columns in series that had been pre-activated with methanol (20 mL) followed by water (120 mL). After loading the product, the columns were washed with water (50 mL), purged with air, and the product was eluted with methanol (10 mL). The filtrate was directly concentrated by rotary evaporation and trace solvent was removed by high vacuum (2 hours) affording a white semi-solid (21.2 mg, 54%). The solid was stored as a 5 mM solution in 20 mM NaCl+10% DMA at −18° C.

HPLC-analysis and mass spectrometry showed that the product was 95% pure.

HRMS (ESI⁺) C₅₅H₁₀₁ClN₁₁O₁₀Pt [M+H]⁺ calc 1304.7043, found 1304.6968.

### Synthesis of Auristatin-Maleimide (AF-Mal)

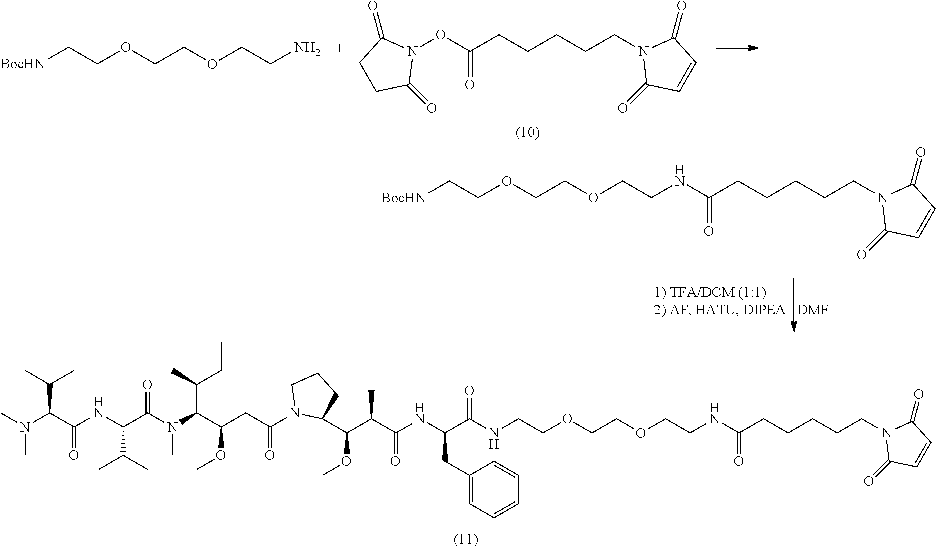

### tert-butyl(2-(2-(2-(6-(2,5-dioxo-2,5-dihydro-1H-pyrrolyl) hexanamido) ethoxy) ethoxy) ethyl) carbamate (BocNH-PEG₁-amide-Hex-Mal) (10)

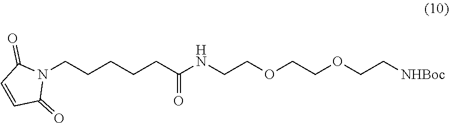 [SMILES: CC(C)(C)OC(=O)NCCOCCOCCNC(=O)CCCCCN1C(=O)C=CC1=O]

2,5-dioxopyrrolidin-1-yl 6-(2,5-dioxo-2,5-dihydro-1H-pyrrol-1-yl)hexanoate (62.1 mg. 0.201 mmol) was dissolved in DMF (1 mL) and tert-butyl (2-(2-(2 aminoethoxy)ethoxy)ethyl)carbamate (50 mg, 0.201 mmol) in DMF (1 mL) was added dropwise under argon, followed by the addition of DIPEA (0.053 ml, 0.302 mmol). The reaction was stirred for 40 hours at room temperature and subsequently concentrated on silica. Purification by flash chromatography (1-4% MeOH in DCM) afforded the product (10) (56 mg, 63%) as a colorless oil.

¹H-NMR (500 MHz, CDCl₃) δ 6.66 (s, 2H), 6.05 (br s, 1H), 5.02 (br s, 1H), 3.70-3.36 (m, 12H), 3.36-3.20 (m, 2H), 2.15 (t, J=7.4 Hz, 2H), 1.66-1.54 (m, 4H), 1.42 (s, 9H), 1.31-1.25 (m, 2H).

¹³C-NMR (500 MHz, CDCl₃) δ 172.7, 170.8, 155.9, 134.0, 79.3, 70.2, 70.1, 70.1, 69.9, 40.2, 39.1, 37.6, 36.3, 28.3, 28.2, 26.3, 25.0.

HRMS (ESI⁺) C₂₁H₃₅N₃O₇ [M+Na]⁺ calc 464.2367, found 464.2363.

AF-PEG₁-amide-Hex-Mal (11)

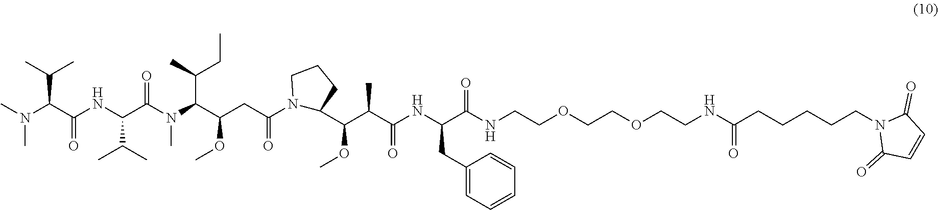 [SMILES: CC[C@H](C)[C@@H]([C@@H](CC(=O)N1CCC[C@H]1[C@H](OC)[C@@H](C)C(=O)N[C@H](Cc1ccccc1)C(=O)NCCOCCOCCNC(=O)CCCCCN1C(=O)C=CC1=O)OC)N(C)C(=O)[C@@H](NC(=O)[C@H](C(C)C)N(C)C)C(C)C]

(10) (11.25 mg, 0.025 mmol) was dissolved in DCM (0.15 mL) and TFA (0.15 mL) was added to the solution. The mixture was stirred for 105 minutes followed by evaporation under a nitrogen flow and subsequent high vacuum (1 hour). A solution of AF (19.0 mg, 0.025 mmol) in DMF (1 ml) was then added to the vial containing the crude reaction mixture, after which HATU (19.37 mg, 0.051 mmol) and DIPEA (0.013 ml, 0.076 mmol) were consecutively added. The mixture was stirred for 2 hours at RT under argon, after which the mixture was concentrated. Purification by preparative-HPLC (30-45% MeCN in H₂O in 40 minutes) afforded the product as a colorless solid. The product was stored as a 10 mM solution in DMSO at −18° C.

HPLC-analysis and mass spectrometry showed that the product was 95% pure.

HRMS (ESI⁺) C₅₆H₉₃N₈O₁₂ [M+H]⁺ calc 1069,6908, found 1069.6877; [M+H+Na]²⁺ calc 546.3400, found 546.3368.

The potential of the cis-platin analogue ethylenediamine platinum(II) dichloride, Lx, as a bifunctional linker in the preparation of Antibody-Drug-Conjugates (ADCs) has been explored. The concept of this new linker technology is as follows; in a first step a payload (either tracers or drugs; also referred to as functional moiety) having a suitable coordination group is coordinated to the platinum based linker referred to as “Lx” affording a payload-Lx complex. It has been shown that N-coordinated complexes afforded stable bonds. Interestingly, since the formed platinum complex is charged, the water-solubility of the payload-Lx complex increases tremendously. The conjugation procedure is as follows; the payload-Lx complex is mixed with the antibody solution under slightly basic conditions at 37° C. for 24 hours followed by a post-treatment step with thiourea to remove weakly bound complexes. It has been demonstrated that the formed conjugated were stable in PBS and surface plasmon resonance analysis showed no loss in binding affinity of the antibody after conjugation.

In the present application the potential of Lx as a linker for ADCs is examined. Two Lx payloads (i.e., functional moieties) were designed, namely Desferal-Lx (DFO-Lx) and Auristatin F-Lx (AF-Lx) to study the in vivo behavior and the antitumor effects, respectively. Ultimately, the tumor killing capacity of the ADC determines its commercial success and, therefore, in most studies, the focus is on the antitumor effect. However, the efficacy of the ADC depends strongly on the in vivo stability and tumor targeting capability of the ADC and, therefore, information of the in vivo behavior of the ADC is of importance. Moreover, by studying the in vivo behavior, the fate of the ADC, which does not accumulate in the tumor, is determined, thereby providing information on any potential toxicity risks. The easiest and most reliable way of in vivo characterization is by radiolabeling the ADC like, for example, the introduction of the PET isotope 89Zr in the DFO chelator.

Conjugation of both the DFO-Lx and AF-Lx complex with trastuzumab at 37° C. for 24 hours afforded ADCs, which were 99% monomeric having Drug-Antibody-Ratio (DARs) between 2.5 and 2.7. The versatility of the conjugation method was demonstrated by replacing trastuzumab for obinutuzumab, ofatumumab or IgG-B12 resulting in ADCs with identical characteristics. In the current conjugation procedure, use was made of a 20 fold excess of payload-Lx complex when taking the DAR in consideration the conjugation efficiency is around 13%. The percentage of unconjugated antibody is consistently less than 5% for the conjugates with an average DAR of around 2.5. Moreover, for an average DAR of 2.5 DAR-populations in the range of 2-4 and 1-5 represented 76-78%, respectively, 94-96% of the molecules.

Interestingly, the DAR distribution profile for DFO-Lx-trastuzumab was narrow compared to the lysine conjugated analogue, DFO-trastuzumab, having a comparable average DAR implying that a smaller number of amino acid sites are involved in the Lx conjugation technology.

The coupling position of the Lx-payload in the mAb was determined by separation of the heavy chain and light chain of the antibodies via reduction of the interchain cysteine bridges with DTT. SEC-MS and SDS-PAGE revealed that ca. 90% of the payload was conjugated to the heavy chain. Further investigation with pepsin or papain in order to separate the Fc region from the F(ab)2 or Fab, respectively revealed that ca. 85% was conjugated to the Fc region. This intriguing property of Lx to bind mainly to the Fc part of the mAb is advantageous as it reduces the change of conjugating in the binding region of the mAb thereby affecting the immunoreactivity of the mAb and, therefore, tumor targeting capacity.

The in vitro cell killing of AF-Lx-trastuzumab was tested on a panel of HER2-expressing cell lines. Two benchmarks were included, namely the FDA approved ADC KADCYLA® (T-DM1) and a maleimide analogue AF-Mal-trastuzumab. The latter mimicking the conjugation strategy used by Seattle-Genetics. All three conjugates showed IC50s in the range of 20 to 200 pM for the HER2 over-expressing cell lines SKOV-3, BT-474, NCI-N87 and SK-BR-3. Interestingly, both AF based conjugates showed IC50s in the picomolar range in the trastuzumab and T-DM1 resistant cell line. The AF-Lx was 10³-10⁴ times less toxic compared to AF-Lx-trastuzumab whereas the AF-Mal only was 10 times less toxic than AF-Mal-trastuzumab. Moreover, the AF-Lx is 10² to 10³ times less toxic than AF-Mal.

In vivo stability is an important requirement of an ADC linker. Premature release of the drug form the antibody in the bloodstream results in exposure of the free drug to normal organs leading to unacceptable toxicities. A comparative biodistribution study of ⁸⁹Zr-DFO-Lx-trastuzumab and ⁸⁹Zr-DFO-trastuzumab in nude mice bearing subcutaneous HER2-expressing NCI-N87 xenografts revealed similar ⁸⁹Zr levels in the tumors and all organs for both conjugates with the exception for the liver. This result indicates that the Lx linker technology allows for stable conjugation and efficient tumor targeting. Hydrophobicity and charge of the payload or spacer and number of coupled payloads can have detrimental effects the physicochemical properties of the mAb and consequently on the pharmacokinetic and tumor targeting. Therefore, a second biodistribution study was carried out with ⁸⁹Zr-AF-Lx-trastuzumab and its maleimide conjugated benchmark ⁸⁹Zr-AF-Mal-trastuzumab in nude mice bearing subcutaneous HER2-expressing NCI-N87 xenografts. The uptake of ⁸⁹Zr in the tumor and organs, except for the liver, of the Lx conjugate was similar to ⁸⁹Zr-AF-Lx-trastuzumab and the unconjugated ⁸⁹Zr-trastuzumab. Both biodistribution studies showed a higher liver uptake for the Lx based ADCs compared to the benchmarks.

Finally, the efficacy of AF-Lx-trastuzumab was assessed on two different tumor models, the NCI-N87 and the T-DM1 resistant JIMT-1. The Lx based conjugate outperforms its maleimide benchmark and the FDA approved T-DM1 in both efficacy studies. These results are surprising considering that no significant differences between AF-Lx-trastuzumab and AF-Mal-trastuzumab were observed in either the in vitro cell viability assays or the biodistribution studies suggesting that the improved in vivo efficacy of the auristatin based conjugate is enhanced by Lx.

In conclusion, a new ADC linker technology is shown, which is based on transition metal coordination chemistry of a bifunctional cis-platinum(II) analogue, Lx. The conjugation procedure is robust and straightforward and does not require antibody modification. The formed ADC are stable and physicochemical properties of the native mAb are maintained. Moreover, when combined with Auristatin F, the Lx has an enhancing effect on the antitumor efficacy.

### REFERENCES

1. Jain N., S. W. Smith, S. Ghone, and B. Tomczuk. Current ADC linker chemistry. Pharmaceutical research 2015; 32(11):3526-40.
2. Lambert J. M., and R. V. Chari. Ado-trastuzumab emtansine (T-DM1): An antibody-drug conjugate (ADC) for HER2-positive breast cancer. J. Med. Chem. 2014; 57(16): 6949-64.
3. Senter P. D., and E. L. Sievers. The discovery and development of brentuximab vedotin for use in relapsed Hodgkin lymphoma and systemic anaplastic large cell lymphoma. Nature biotechnology 2012; 30(7):631-37.
4. Chari R. V., M. L. Miller, and W. C. Widdison. Antibody-drug conjugates: an emerging concept in cancer therapy. Angewandte Chemie International Edition 2014; 53(15):3796-827.
5. Ducry L., and B. Stump. Antibody-drug conjugates: linking cytotoxic payloads to monoclonal antibodies. Bioconjugate chemistry 2009; 21(1):5-13.
6. Polakis P. Antibody Drug Conjugates for Cancer Therapy. Pharmacological reviews 2016; 68(1):3-19.
7. Drake P. M., A. E. Albers, J. Baker, S. Banas, R. M. Barfield, A. S. Bhat, et al. Aldehyde tag coupled with HIPS chemistry enables the production of ADCs conjugated site-specifically to different antibody regions with distinct in vivo efficacy and PK outcomes. Bioconjugate chemistry 2014; 25(7): 1331-41.
8. Hamblett K. J., P. D. Senter, D. F. Chace, M. M. Sun, J. Lenox, C. G. Cerveny, et al. Effects of drug loading on the antitumor activity of a monoclonal antibody drug conjugate. Clinical Cancer Research 2004; 10(20): 7063-70.
9. Junutula J. R., H. Raab, S. Clark, S. Bhakta, D. D. Leipold, S. Weir, et al. Site-specific conjugation of a cytotoxic drug to an antibody improves the therapeutic index. Nature biotechnology 2008; 26(8):925-32.
10. Erickson H. K., and J. M. Lambert. ADME of antibody-maytansinoid conjugates. The AAPS journal 2012; 14(4):799-805.
11. Erickson H. K., P. U. Park, W. C. Widdison, Y. V. Kovtun, L. M. Garrett, K. Hoffman, et al. Antibody-maytansinoid conjugates are activated in targeted cancer cells by lysosomal degradation and linker-dependent intracellular processing. Cancer research 2006; 66(8):4426-33.
12. Okeley N. M., J. B. Miyamoto, X. Zhang, R. J. Sanderson, D. R. Benjamin, E. L. Sievers, et al. Intracellular activation of SGN-35, a potent anti-CD30 antibody-drug conjugate. Clinical Cancer Research 2010; 16(3): 888-97.
13. de Goeij B. E., and J. M. Lambert. New developments for antibody-drug conjugate-based therapeutic approaches. Current opinion in immunology 2016; 40:14-23.
14. Dennler P., E. Fischer, and R. Schibli. Antibody Conjugates: From Heterogeneous Populations to Defined Reagents. Antibodies 2015; 4(3):197-224.
15. McCombs J. R., and S. C. Owen. Antibody drug conjugates: design and selection of linker, payload and conjugation chemistry. The AAPS journal 2015; 17(2):339-51.
16. Kim T. H., M. Swierczewska, Y. Oh, A. Kim, D. G. Jo, J. H. Park, et al. Mix to validate: A facile, reversible PEGylation for fast screening of potential therapeutic proteins in vivo. Angewandte Chemie 2013; 125(27):7018-22.
17. Timerbaev A. R., C. G. Hartinger, S. S. Aleksenko, and B. K. Keppler. Interactions of antitumor metallodrugs with serum proteins: advances in characterization using modern analytical methodology. Chemical reviews 2006; 106(6):2224-48.
18. Borch R. F., and M. E. Pleasants. Inhibition of cis-platinum nephrotoxicity by diethyldithiocarbamate rescue in a rat model. Proceedings of the National Academy of Sciences 1979; 76(12):6611-14.
19. Waalboer D. C., J. A. Muns, N. J. Sijbrandi, R. Schasfoort, R. Haselberg, G. W. Somsen, et al. Platinum (II) as Bifunctional Linker in Antibody-Drug Conjugate Formation: Coupling of a 4-Nitrobenzo-2-oxa-1,3-diazole Fluorophore to Trastuzumab as a Model. Chem. Med. Chem. 2015; 10(5):797-803.
20. Banci L., I. Bertini, O. Blaževitš, V. Calderone, F. Cantini, J. Mao, et al. Interaction of cisplatin with human superoxide dismutase. Journal of the American Chemical Society 2012; 134(16):7009-14.
21. Calderone V., A. Casini, S. Mangani, L. Messori, and P. L. Orioli. Structural investigation of cisplatin-protein interactions: selective platination of His19 in a cuprozinc superoxide dismutase. Angewandte Chemie International Edition 2006; 45(8):1267-69.
22. Casini A., G. Mastrobuoni, C. Temperini, C. Gabbiani, S. Francese, G. Moneti, et al. ESI mass spectrometry and X-ray diffraction studies of adducts between anticancer platinum drugs and hen egg white lysozyme. Chem. Commun. 2006 (2):156-58.
23. Ferraro G., L. Massai, L. Messori, and A. Merlino. Cisplatin binding to human serum albumin: a structural study. Chemical Communications 2015; 51(46):9436-39.
24. Messori L., T. Marzo, E. Michelucci, I. Russo Krauss, C. Navarro-Ranninger, A. G. Quiroga, et al. Interactions between Anticancer trans-Platinum Compounds and Proteins: Crystal Structures and ESI-MS Spectra of Two Protein Adducts of trans-(Dimethylamino)(methyl amino) dichloridoplatinum (II). Inorganic chemistry 2014; 53(15):7806-08.
25. Vugts D. J., G. W. Visser, and G. A. van Dongen. 89Zr-PET radiochemistry in the development and application of therapeutic monoclonal antibodies and other biologicals. Current topics in medicinal chemistry 2013; 13(4):446-57.
26. Verel I., G. W. Visser, R. Boellaard, M. Stigter-van Walsum, G. B. Snow, and G. A. van Dongen. 89Zr immuno-PET: comprehensive procedures for the production of 89Zr-labeled monoclonal antibodies. Journal of Nuclear Medicine 2003; 44(8):1271-81.
27. Tanner M., A. I. Kapanen, T. Junttila, O. Raheem, S. Grenman, J. Elo, et al. Characterization of a novel cell line established from a patient with Herceptin-resistant breast cancer. Molecular cancer therapeutics 2004; 3 (12): 1585-92.
28. Lindmo T., E. Boven, F. Cuttitta, J. Fedorko, and P. Bunn. Determination of the immunoreactive function of radiolabeled monoclonal antibodies by linear extrapolation to binding at infinite antigen excess. Journal of immunological methods 1984; 72(1):77-89.
29. Axup J. Y., K. M. Bajjuri, M. Ritland, B. M. Hutchins, C. H. Kim, S. A. Kazane, et al. Synthesis of site-specific antibody-drug conjugates using unnatural amino acids. Proceedings of the National Academy of Sciences 2012; 109(40):16101-06.
30. Doronina S. O., B. A. Mendelsohn, T. D. Bovee, C. G. Cerveny, S. C. Alley, D. L. Meyer, et al. Enhanced activity of monomethylauristatin F through monoclonal antibody delivery: effects of linker technology on efficacy and toxicity. Bioconjugate chemistry 2006; 17(1):114-24.
31. Barok M., H. Joensuu, and J. Isola. Trastuzumab emtansine: mechanisms of action and drug resistance. Breast cancer research: BCR 2014; 16(2):209.
32. Koninki K., M. Barok, M. Tanner, S. Staff, J. Pitkänen, P. Hemmilä, et al. Multiple molecular mechanisms underlying trastuzumab and lapatinib resistance in JIMT-1 breast cancer cells. Cancer letters 2010; 294(2):211-19.
33. Loganzo F., X. Tan, M. Sung, G. Jin, J. S. Myers, E. Melamud, et al. Tumor Cells Chronically Treated with a Trastuzumab-Maytansinoid Antibody-Drug Conjugate Develop Varied Resistance Mechanisms but Respond to Alternate Treatments. Molecular cancer therapeutics 2015; 14(4): 952-63.
34. Živković M. D., S. Rajković, and M. I. Djuran. Reaction of [Pt (Gly-Gly-N, N′, 0) I]— with the N-acetylated dipeptide 1-methionyl-1-histidine: Selective platination of the histidine side chain by intramolecular migration of the platinum (II) complex. Bioorganic Chemistry 2008; 36(3):161-64.
35. Boelrijk A. E., P. J. Boogaard, E. L. Lempers, and J. Reedijk. Regeneration experiments of the platinated enzyme fumarase, using sodium diethyldithiocarbamate, thiourea, and sodium thiosulfate. Journal of inorganic biochemistry 1991; 41(1):17-24.
36. Deubel D. V. On the competition of the purine bases, functionalities of peptide side chains, and protecting agents for the coordination sites of dicationic cisplatin derivatives. Journal of the American Chemical Society 2002; 124(20):5834-42.
37. Boswell C. A., D. B. Tesar, K. Mukhyala, F-P Theil, P. J. Fielder, and L. A. Khawli. Effects of charge on antibody tissue distribution and pharmacokinetics. Bioconjugate chemistry 2010; 21(12):2153-63.
38. Igawa T., H. Tsunoda, T. Tachibana, A. Maeda, F. Mimoto, C. Moriyama, et al. Reduced elimination of IgG antibodies by engineering the variable region. Protein Engineering Design and Selection 2010; 23(5):385-92.
39. Roopenian D. C., and S. Akilesh. FcRn: the neonatal Fc receptor comes of age. Nature Reviews Immunology 2007; 7(9):715-25.

1. Cell targeting conjugates, which cell targeting conjugates comprise a targeting moiety and one or more functional moieties bound thereto via a linker,
wherein the linker comprises a transition metal complex and
wherein at least 90% of the conjugates have a ratio of functional moieties to targeting moieties (DAR) of 4 or less.

2. Cell targeting conjugates according to claim 1, wherein at least 50% of the conjugates have a ratio of functional moieties to targeting moieties (DAR) of 2 or 3.

3. Cell targeting conjugates according to claim 1, wherein the targeting moiety is a peptide, an antibody, an antibody fragment or a nanobody.

4. Cell targeting conjugates according to claim 1, wherein conjugation of the functional moieties to the targeting moiety is performed without modification of the targeting moiety.

5. Cell targeting conjugates according to claim 1, wherein at least 70% of the functional moieties are bound to the Fc part of the targeting moiety.

6. Cell targeting conjugates, which cell targeting conjugates comprise a targeting moiety and one or more functional moieties bound thereto via a linker,
wherein the linker comprises a transition metal complex and the targeting moiety comprises an antibody, and
wherein at least 70% of the functional moieties are bound to the Fc part of the antibody.

7. Cell targeting conjugates according to claim 6, wherein at least 80% of the functional moieties are bound to the Fc part of the antibody.

8. Cell targeting conjugates according to claim 1, wherein the linker comprises a platinum complex.

9. Cell targeting conjugates according to claim 1, wherein the linker comprises a bidentate moiety.

10. Cell targeting conjugates according to claim 1, wherein the functional moiety is a therapeutic compound, a diagnostic compound and/or a chelating agent.

11. Cell targeting conjugates according to claim 1,
wherein the targeting moiety is trastuzumab, obinituzumab or ofatumumab, and
wherein the functional moiety is selected from the group consisting of a supertoxic drug, desferrioxamine (DFO), auristatin F (AF) and a kinase inhibitor.

12. Pharmaceutical composition comprising:
the cell targeting conjugates claim 1, and
a pharmaceutically acceptable carrier.

13. Pharmaceutical composition comprising
a pharmaceutically acceptable carrier and
cell targeting conjugates, which conjugates comprise a targeting moiety and one or more functional moieties bound thereto via a linker,
wherein the linker comprises a transition metal complex and
wherein at least 90% of the conjugates have a ratio of functional moieties to targeting moieties (DAR) of 4 or lower.

14. Pharmaceutical composition according to claim 13, wherein at least 50% of the conjugates have a ratio of functional moieties to targeting moieties (DAR) of 2 or 3.

15. Pharmaceutical composition according to claim 13, wherein the linker of the conjugates comprises a platinum complex.

16. Pharmaceutical composition according to claim 13, wherein the linker of the conjugates comprises a bidentate moiety.

17. Pharmaceutical composition according to claim 13, wherein the targeting moiety of the conjugates is a peptide, an antibody, an antibody fragment or a nanobody.

18. Pharmaceutical composition according claim 17, wherein conjugation of the functional moieties to the targeting moiety is performed without modification of the targeting moiety.

19. Pharmaceutical composition according to claim 17, wherein at least 70% of the functional moieties are bound to the Fc part of the targeting moiety.

20. Pharmaceutical composition according to claim 13, wherein the functional moiety of the conjugates is a therapeutic compound, a diagnostic compound and/or a chelating agent.

21. Pharmaceutical composition according to claim 13,
wherein the targeting moiety is trastuzumab, obinituzumab or ofatumumab, and
wherein the functional moiety is selected from the group consisting of a supertoxic drug, desferrioxamine (DFO), auristatin F (AF) and a kinase inhibitor.

22. (canceled)

23. A method of treating a subject for cancer, the method comprising:
administering to the subject the cell targeting conjugates of claim 1.

24. The method according to claim 23, wherein the cancer is breast cancer or stomach cancer.

25. Method for the treatment of cancer comprising administering to a patient in need thereof a pharmaceutically effective amount of the pharmaceutical composition according to claim 21.

26. Method according to claim 25, wherein the cancer is breast cancer or stomach cancer.

27. A method of treating a cancer cell, the method comprising:
contacting said cancer cell with cell targeting conjugates, which cell targeting conjugates comprise a targeting moiety and one or more functional moieties bound thereto via a linker, wherein the linker comprises a transition metal complex.

28. The method according to claim 27, wherein the cancer cell is breast cancer or stomach cancer.

29. The method according to claim 28, wherein the linker of the cell targeting conjugates comprises a platinum complex.
